(19) United States
(12) Patent Application Publication
(10) Pub. No.: US 2025/0275536 A1
(43) Pub. Date: Sep. 4, 2025

(54) Antimicrobial Composition

(71) Applicant: Patrick Kamer, Wädenswil (CH)

(72) Inventors: Patrick Kamer, Wädenswil (CH); Annette Rees, Zug (CH)

(21) Appl. No.: 18/862,514

(22) PCT Filed: May 3, 2023
(86) PCT No.: PCT/EP2023/061616

§ 371 Date: Nov. 1, 2024

(30) Foreign Application Priority Data
May 3, 2022 (EP) 22171476.9

(51) Int. Cl. A01N 33/12; A01N 25/02; A01P 1/00

(52) U.S. Cl. CPC A01N 33/12; A01N 25/02; A01P 1/00

(57) ABSTRACT

The present invention relates to a composition comprising or consisting of a specific quaternary compound, an alcohol or polyol, an inorganic or organic acid and an essential oil. The invention also relates to a disinfectant or an antimicrobial preparation comprising said composition. Additionally, the present invention relates to the use of the composition for sanitizing or disinfecting a surface, skin, or an object. Moreover, the present invention pertains to a method for sanitizing or disinfecting a surface, skin, or an object. Finally, the present invention relates to a method for preparing the specific quaternary compounds.

### TECHNICAL FIELD

The present invention relates to a composition comprising or consisting of a specific quaternary compound, an alcohol or polyol, an inorganic or organic acid and an essential oil. The invention also relates to a disinfectant or an antimicrobial preparation comprising said composition. Additionally, the present invention relates to the use of the composition for sanitizing or disinfecting a surface, skin, or an object. Moreover, the present invention pertains to a method for sanitizing or disinfecting a surface, skin, or an object. Finally, the present invention relates to a method for preparing the specific quaternary compounds.

### BACKGROUND ART

Our planet and human society are facing devastating challenges regarding global health threats from viral and bacterial infections causing tremendous healthcare costs and mortality. Similarly, fungal, and parasitic infections affect humans. Moreover, environmental challenges are further huge problems we are encountering. In this context, the invention of innovative environmentally friendly technologies for battling these huge challenges is critically required.

Recently, we were confronted with these challenges in the worst global pandemic, the coronavirus disease 2019 (COVID-19), caused by the novel severe acute respiratory syndrome coronavirus 2 (SARS-Cov-2), which have impacted our society tremendously leading to humanitarian and economic crises. This incident will lead to a paradigm shift in our society and daily lifestyle, resulting in precaution and improved hygienic routine in various domestic and public settings. Especially, retails and hospitals, public spaces and transportations, schools and governmental agencies, food and packaging.

A modern hygiene concept should meet the following requirements:
a broad-spectrum effect that actually renders all pathogenic germs harmless with just one formulation;
a low toxicity for humans, but also animals and the environment;
develops its full effect with the shortest possible exposure time; and
ideally retains its full duration of action for two days and beyond.

The general means of reducing the spreading of microorganisms and viruses are through hand washing and effective sanitizing. However, most sanitizing and disinfectant products have a short lifetime and only prevent the spreading of the microorganism or viruses or killing these at the time of use.

There is a significant number of surface disinfectants containing components such as triclosan, however, this compound has created several human health concerns related to microbial resistance. Alcohol-based disinfectants are known to display antimicrobial activity, nevertheless, their fast evaporation limits their application. Chlorine-based disinfectants are also another class of disinfectants widely used, but they might be toxic and lead to corrosion. Quaternary ammonium-based disinfectants are also employed and have shown to display high antimicrobial activity. Nonetheless, they might be toxic and have certain limited activity against some types of microbes.

In addition, one of the main limitations with the commercially available disinfectant is that they only function upon application, thus effectively killing microbes immediately on the surface, for a limited amount of time, nevertheless, surfaces can easily and rapidly be re-contaminated since the antimicrobial activity of most disinfectants does not last for longer periods of time.

Known biocidal formulations not based on alcohols or polyols contain one or more different quaternary organic ammonium compounds as the main biocidal substance(s), and their formulation systems have so far generated the following disadvantages: These biocidal formulations are only sufficiently biocidal against the entire microbial spectrum (such as enveloped viruses, normal viruses, yeasts and moulds, spores, encapsulated spores, as well as against all bacterial species) if additional different quaternary organic ammonium compounds are contained in the biocidal formulation and the total amount of quaternary organic ammonium compounds is included in highly harmful concentration, which is environmentally toxic, orally toxic, as well as dermally harmful. In addition, most of these quaternary organic ammonium compound formulations provide no or insufficient, long-term retarding biocidal activity against the entire microbial spectrum, and, thus, must be repeatedly sprayed or applied several times a day, in order to sufficiently prevent microbial contamination in accordance with the European biocide regulation requirement.

The use of a mixture of N1-hexadecyl-N1,N1,N2,N2-tetramethyl-N2-(3-(trihyroxysilyl-)propyl-)ethane-1,2-diaminium dichloride, N1,N1,N2,N2-Tetramethyl-N1-octadecyl-N2-(3-(trihydroxysilyl-)propyl-)ethane-1,2-diamonium dichloride and octadecyldimethyl-(3-trihydroxysilylpropyl-)ammonium chloride in a hand sanitizer is already known. The only proven long-term retarding but limited broad-spectrum biocidal formulation however has the disadvantage, that it is only effective against the entire microbial spectrum when it is used in combination with high concentrations of additional differently structured quaternary organic ammonium compounds. Said known hand sanitizer shows a long-lasting antimicrobial effect only due to a high content of benzalkonium chloride (0.13 wt.-%), lauramine oxide (0.13 wt.-%) and didecyldimethylammoniumchloride (0.5 wt.-%), i.e. the total content of these quaternary organic ammonium compounds amounts to 0.76 wt.-% in the sanitizer.

However, benzalkonium chloride, lauramine oxide and didecyldimethylammoniumchloride are highly toxic and irritant to human skin, eyes, to animals and to the environment. They are highly toxic to respiratory and gastrointestinal organs, to the immune system and to nervous tissue. Due to the high content of the quaternary organic ammonium compounds of 0.85 wt.-% in total, which exceeds the maximum permissible value, said commercially available hand sanitizer does not comply with the EU biocide regulation requirements.

Thus, more than ever, there is still an ongoing need for innovative and environmentally friendly long-lasting solutions that prevent and limit the spread of bacteria, viruses, fungal and parasitic organisms in various public, industrial, domestic, professional, and academic settings.

It is therefore an object of the present invention to overcome the above-mentioned problems and to provide an antimicrobial composition having a broad-spectrum effect that actually renders all pathogenic germs harmless with just one formulation, having a low toxicity for humans, but also animals and the environment; developing its full effect with the shortest possible exposure time; and ideally retaining its full duration of action for two days and beyond.

Herein, is disclosed a multifunctional environmentally friendly antimicrobial, i.e. antibacterial, antiviral, antifungal, and antiparasitic, composition for disinfecting surfaces, skin and objection with long-lasting activity. The presented invention kills a wide range of bacteria, viruses, fungal, and parasite organisms immediately after application and continues to protect the surface, the skin or an object and kills these organisms for at least two days. The present invention thus prevents re-contamination of surfaces. The facile fabrication approach comprises by simply mixing the composition ingredients with an appropriate solvent without the use of toxic ingredients. The composition can easily be applied through various application methods such as spraying or wiping.

Surprisingly it has been found that the composition according to the present invention as defined exhibits a long-lasting and broad antimicrobial spectrum effect against gram-positive bacteria, gram-negative bacteria, viruses, non-enveloped and enveloped viruses, mould, mildew, yeast, fungi, and parasites despite a low content of quaternary organic ammonium compounds.

### SUMMARY OF THE INVENTION

To accomplish the above problem, the present invention provides in a first aspect for a composition, comprising or consisting of
(a) at least one quaternary compound represented by the general formula (I)

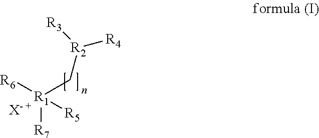

wherein
   R₂ and R₂ are independently from each other selected from the group consisting of nitrogen and phosphor;
   R₃ and R₄ are independently from each other selected from the group consisting of H, C1 to C36 n-alkyl, C1 to C36 i-alkyl, C1 to C36 sec-alkyl, C1 to C36 t-alkyl, aryl or C1 to C36 t-alkyl-silyl;
   R₅ and R₆ are independently from each other selected from the group consisting of H, C1 to C36 n-alkyl, C1 to C36 i-alkyl, C1 to C36 sec-alkyl, C1 to C36 t-alkyl, aryl and C1 to C36 t-alkyl-silyl;
   R₇ is a C1 to C36 n-alkyl;
   X is selected from the group consisting of Cl, Br, F, I, HSO₄, 0.5HPO₄, H₂PO₄, 0.5CO₃, 0.5SO₄, ⅓PO₄, CH₃SO₃, CF₃SO₃, NTf₂, BF₄, acetate, tosylate, benzoate, salicylate, succinate, saccharinate, —OH and gluconate; and
   n is an integer in a range from 1 to 36;
or at least one quaternary compound represented by the general formula (II)

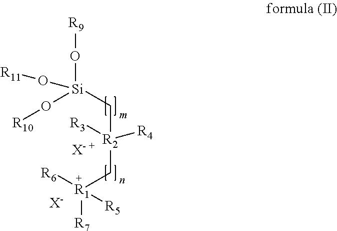

wherein
   R₁ and R₂ are independently from each other selected from the group consisting of nitrogen and phosphor;
   R₃ and R₄ are independently from each other selected from the group consisting of H, C1 to C36 n-alkyl, C1 to C36 i-alkyl, C1 to C36 sec-alkyl, C1 to C36 t-alkyl, aryl or C1 to C36 t-alkyl-silyl;
   R₅ and R₆ are independently from each other selected from the group consisting of H, C1 to C36 n-alkyl, C1 to C36 i-alkyl, C1 to C36 sec-alkyl, C1 to C36 t-alkyl, aryl and C1 to C36 t-alkyl-silyl;
   R₇ is a C1 to C36 n-alkyl;
   R₉, R₁₀ and R₁₁ are independently from each other selected from the group consisting of H, C1 to C36 n-alkyl, C1 to C36 i-alkyl, C1 to C36 sec-alkyl, C1 to C36 t-alkyl and aryl;
   X is selected from the group consisting of Cl, Br, F, I, HSO₄, 0.5HPO₄, H₂PO₄, 0.5CO₃, 0.5SO₄, ⅓PO₄, CH₃SO₃, CF₃SO₃, NTf₂, BF₄, acetate, tosylate, benzoate, salicylate, succinate, saccharinate, —OH and gluconate;
   n is an integer in a range from 1 to 36; and
   m is an integer in a range from 1 to 36;
(b) at least one alcohol and/or polyol;
(c) at least one inorganic and/or organic acid or a salt or a derivative thereof; and
(d) at least one essential oil.

In a second aspect, the present invention provides for a disinfectant or an antimicrobial preparation comprising the composition according to the present invention.

In a further aspect, the present invention provides for the use of said composition or the disinfectant or antimicrobial preparation for disinfecting, for disinfecting surfaces, skin, or objects.

In a still further aspect, the present invention provides for a method for disinfecting a surface, skin or an object comprising the steps of:
applying an effective amount of the composition as defined herein or the disinfectant, antimicrobial cosmetic or pharmaceutical preparation as defined herein onto a surface, skin, or object; and
contacting the surface, skin or the object with the composition, disinfectant, or preparation for a contact time of at least 15 seconds, preferably at least 30 minutes.

Finally, the present invention provides for a method for preparing a quaternary compound according to general formula (I) or the general formula (II) as defined herein.

### DETAILED DESCRIPTION OF THE INVENTION

The present invention is specified in the appended claims. The invention itself, and its preferred variants, other objects and advantages, are however also apparent from the following detailed description in conjunction with the accompanying examples and figures.

In a first aspect, the present invention relates to a composition, comprising or consisting of
(a) at least one quaternary compound represented by the general formula (I)

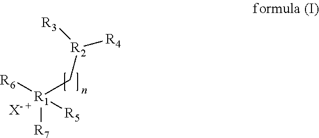

wherein
   R₁ and R₂ are independently from each other selected from the group consisting of nitrogen and phosphor;
   R₃ and R₄ are independently from each other selected from the group consisting of H, C1 to C36 n-alkyl, C1 to C36 i-alkyl, C1 to C36 sec-alkyl, C1 to C36 t-alkyl, aryl or C1 to C36 t-alkyl-silyl;
   R₅ and R₆ are independently from each other selected from the group consisting of H, C1 to C36 n-alkyl, C1 to C36 i-alkyl, C1 to C36 sec-alkyl, C1 to C36 t-alkyl, aryl and C1 to C36 t-alkyl-silyl;
   R₇ is a C1 to C36 n-alkyl;
   X is selected from the group consisting of Cl, Br, F, I, HSO₄, 0.5HPO₄, H₂PO₄, 0.5CO₃, 0.5SO₄, ⅓PO₄, CH₃SO₃, CF₃SO₃, NTf₂, BF₄, acetate, tosylate, benzoate, salicylate, succinate, saccharinate, —OH and gluconate; and
   n is an integer in a range from 1 to 36;
or at least one quaternary compound represented by the general formula (II)

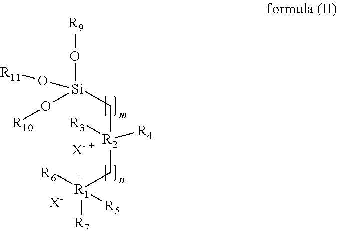

wherein
   R₁ and R₂ are independently from each other selected from the group consisting of nitrogen and phosphor;
   R₃ and R₄ are independently from each other selected from the group consisting of H, C1 to C36 n-alkyl, C1 to C36 i-alkyl, C1 to C36 sec-alkyl, C1 to C36 t-alkyl, aryl or C1 to C36 t-alkyl-silyl;
   R₅ and R₆ are independently from each other selected from the group consisting of H, C1 to C36 n-alkyl, C1 to C36 i-alkyl, C1 to C36 sec-alkyl, C1 to C36 t-alkyl, aryl and C1 to C36 t-alkyl-silyl;
R₇ is a C1 to C36 n-alkyl;
R₉, R₁₀ and R₁₁ are independently from each other selected from the group consisting of H, C1 to C36 n-alkyl, C1 to C36 i-alkyl, C1 to C36 sec-alkyl, C1 to C36 t-alkyl and aryl;
X is selected from the group consisting of Cl, Br, F, I, HSO₄, 0.5HPO₄, H₂PO₄, 0.5CO₃, 0.5SO₄, ⅓PO₄, CH₃SO₃, CF₃SO₃, NTf₂, BF₄, acetate, tosylate, benzoate, salicylate, succinate, saccharinate, —OH and gluconate;
   n is an integer in a range from 1 to 36; and
   m is an integer in a range from 1 to 36;
(b) at least one alcohol and/or polyol;
(c) at least one inorganic and/or organic acid or a salt or a derivative thereof; and
(d) at least one essential oil.

The term “comprising” means that the named components are essential, but other components may be added and is still embraced by the present invention.

The term “consisting of” as used according to the present invention means that the total amount of components (a) to (d) adds up to 100 wt.-%, based on the total weight of the composition, and signifies that the subject matter is closed-ended and can only include the limitations that are expressly recited.

Whenever reference is made to “comprising” it is intended to cover both meanings as alternatives, that is the meaning can be either “comprising” or “consisting of”, unless the context dictates otherwise.

The term “at least one . . . ” as used herein means that the composition according to the present invention can comprise either one of the subsequently described component/compound/Ingredient or a mixture of two, three, four, five, six or even more different of said component/compound/ingredient.

The term “optionally” means that the subsequently described compound may but need not to be present in the composition, and that the description includes variants of compositions or preparations, where the compound/component/ingredient is included or variants, where the compound/component/ingredient is absent.

The component (a) in the composition according to the first aspect of the present invention is in a first alternative at least one quaternary compound represented by the general formula (I)

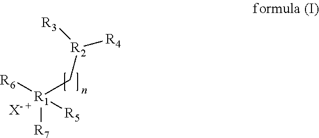

wherein
   R₁ and R₂ are independently from each other selected from the group consisting of nitrogen and phosphor;
   R₃ and R₄ are independently from each other selected from the group consisting of H, C1 to C36 n-alkyl, C1 to C36 i-alkyl, C1 to C36 sec-alkyl, C1 to C36 t-alkyl, aryl or C1 to C36 t-alkyl-silyl;
   R₅ and R₆ are independently from each other selected from the group consisting of H, C1 to C36 n-alkyl, C1 to C36 i-alkyl, C1 to C36 sec-alkyl, C1 to C36 t-alkyl, aryl and C1 to C36 t-alkyl-silyl;
   R₇ is a C1 to C36 n-alkyl;
   X is selected from the group consisting of Cl, Br, F, I, HSO₄, 0.5HPO₄, H₂PO₄, 0.5CO₃, 0.5SO₄, ⅓PO₄, CH₃SO₃, CF₃SO₃, NTf₂, BF₄, acetate, tosylate, benzoate, salicylate, succinate, saccharinate, —OH and gluconate; and
   n is an integer in a range from 1 to 36.

The term “alkyl” or “alkyl groups” in the context of the present invention and as used herein refers to saturated hydrocarbons having one or more carbon atoms, including straight chain monovalent alkyl groups (e.g. methyl, ethyl, propyl, butyl, pentyl, hexyl, heptyl, octyl, nonyl, decyl, etc.), branched chain monovalent alkyl groups (e.g. isopropyl, tert-butyl, sec-butyl, isobutyl, etc.) or cyclic alkyl groups (cycloalkyl) (e.g. cyclopropyl, cyclopentyl, cyclohexyl, cycloheptyl, cyclooctyl, etc.). In a preferred variant the alkyl group is a straight chain or a branched chain alkyl group.

Unless otherwise specified, the term “alkyl” includes both unsubstituted or substituted alkyl. The term “substituted alkyls” refers to alkyl groups having substituents replacing one or more hydrogens on one or more carbons of the hydrocarbon backbone. Such substituents may include, for example, alkenyl, alkynyl, halogeno, hydroxyl, alkylcarbonyloxy, arylcarbonyloxy, alkyoxycrabonyloxy, aryloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkyoxyl, phosphate, phosphonato, phospinato, cyano, amino (including alkyl amino, dialkylamino, arylamino, diarylamino and alkylarylamino), acylamio (including alkylcarbonylamino, arylcarbonylamino, carbamoyl and ureido), imino, sulfydryl, alkylthio, arylthiol, thiocarboxylate, sulfates, alkylsulfinyl, sulfonates, sulfamoyl, sulfonamido, nitro, trifluormethyl, cyano, azido, heterocylic, alkylaryl or aromatic (including heteroaromatic groups).

In some variants, substituted alkyls can include a heterocyclic group. As used herein, the term “heterocyclic group” includes closed ring structures analogous to carbocyclic groups in which one or more of the carbon atoms in the ring is an element other than carbon, for example, nitrogen, sulphur or oxygen. Heterocyclic groups may be saturated or unsaturated. Exemplary heterocyclic groups include, but are not limited to, aziridine, ethylene oxide (epoxides, oxiranes), thiirane (episulfides), dioxirane, azetidine, oxetane, thietane, dioxetane, dithietane, dithiete, azolidine, pyrrolidine, pyrroline, oxolane, dihydrofuran, and furan.

Preferably, the alkyl groups according to the present invention are unsubstituted.

Alkyl groups generally includes those with 1 to 36 carbon atoms. Preferred examples of “alkyl” as used herein include C1 to C36 alkyl chains, i.e. the alkyl is selected from the group consisting of C1 alkyl, C2 alkyl, C3 alkyl, C4 alkyl, C5 alkyl, C6 alkyl, C7 alkyl, C8 alkyl, C9 alkyl, C10 alkyl, C10 alkyl, C11 alkyl, C12 alkyl, C13 alkyl, C14 alkyl, C15 alkyl, C16 alkyl, C17 alkyl, C18 alkyl, C19 alkyl, C20 alkyl. C21 alkyl, C22 alkyl, C23 alkyl. C24 alkyl, C25 alkyl, C26 alkyl, C27 alkyl, C28 alkyl, C29 alkyl, C30 alkyl, C31 alkyl, C32 alkyl, C33 alkyl, C34 alkyl, C35 alkyl and C36 alkyl.

In addition, the term “alkyl” may include “alkenylene”. The term “alkenylenes” refers to a straight chain, branched or cyclic alkyl group having one or more carbon-carbon double bonds and further including at least one double bond. Alkenylene groups generally include those with 1 to 36 carbon atoms. The alkylene groups may be unsubstituted or substituted with those substituents similarly as described for the alkyl groups.

As used herein, “aryl” or “aromatic” groups are cyclic aromatic hydrocarbons that do not contains heteroatoms. Aryl groups include monocyclic, bicyclic, and poly cyclic ring systems. Thus, aryl groups include, but are not limited to, phenyl, azulenyl, heptalenyl, biphenylenyl, indacenyl, florenyl, phenanthrenyl, triphenylenyl, pyrenyl, naphthacenyl, chrysenyl, biphenyl, anthracenyl, indenyl, indanyl, pentalenyl, and naphthyl groups. In some embodiments, aryl groups contain 6 to 14 carbons, in others from 6 to 12 or 6 to 10 carbon atoms in the ring portions of the groups. The phrase “aryl groups” includes groups containing fused rings, such as fused aromatic-aliphatic ring systems. Aryl groups may be unsubstituted or substituted with those substituents similarly as described for the alkyl groups.

In a preferred variant according to the first aspect of the present invention in general formula (I)
R₁ and R₂ are independently from each other selected from the group consisting of nitrogen and phosphor; and/or
R₃ and R₄ are independently from each other selected from the group consisting of H—, methyl, ethyl, n-propyl, i-propyl, n-butyl and sec-butyl; and/or
R₅ and R₆ are independently from each other selected from the group consisting of H—, methyl, ethyl, n-propyl, i-propyl n-butyl and sec-butyl; and/or
R₇ is a C1 to C36 n-alkyl; and/or
X is selected from the group consisting of Cl, Br, F, I, HSO₄, 0.5HPO₄, H₂PO₄, 0.5CO₃, 0.5SO₄, ⅓PO₄, CH₃SO₃, CF₃SO₃, NTf₂, BF₄, acetate, tosylate, benzoate, salicylate, succinate, saccharinate, —OH and gluconate; and/or
n is an integer in a range from 1 to 36;

In a more preferred variant according to the first aspect of the present invention, in general formula (I)
R₁ and R₂ are independently from each other selected from the group consisting of nitrogen and phosphor; and/or
R₃ and R₄ are independently from each other selected from the group consisting of methyl, ethyl, n-propyl, i-propyl, and n-butyl; and/or
R₅ and R₆ are independently from each other selected from the group consisting of methyl, ethyl and n-propyl; and/or
R₇ is a C6 to C24 n-alkyl; and/or
X is selected from the group consisting of Cl, Br, F and I; and/or
n is an integer from 10 to 20.

In a most preferred variant according to the first aspect of the present invention in general formula (I)
R₁ and R₂ are nitrogen; and/or
R₃ and R₄ are independently from each other selected from the group consisting of methyl and ethyl; and/or
R₅ and R₆ are independently from each other selected from the group consisting of methyl and ethyl; and/or
R₇ is a C10 to C20 n-alkyl; and/or
X is selected from the group consisting of Cl and Br; and/or
n is an integer from 12 to 18;

The component (a) in the composition according to the first aspect of the present invention is in a second alternative at least a quaternary compound represented by the general formula (II)

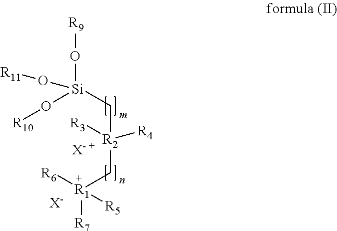

wherein
   R₁ and R₂ are independently from each other selected from the group consisting of nitrogen and phosphor;
   R₃ and R₄ are independently from each other selected from the group consisting of H, C1 to C36 n-alkyl, C1 to C36 i-alkyl, C1 to C36 sec-alkyl, C1 to C36 t-alkyl, aryl or C1 to C36 t-alkyl-silyl;
   R₅ and R₆ are independently from each other selected from the group consisting of H, C1 to C36 n-alkyl, C1 to C36 i-alkyl, C1 to C36 sec-alkyl, C1 to C36 t-alkyl, aryl and C1 to C36 t-alkyl-silyl;
   R₇ is a C1 to C36 n-alkyl;
   R₉, R₁₀ and R₁₁ are independently from each other selected from the group consisting of H, C1 to C36 n-alkyl, C1 to C36 i-alkyl, C1 to C36 sec-alkyl, C1 to C36 t-alkyl and aryl;
   X is selected from the group consisting of Cl, Br, F, I, HSO₄, 0.5HPO₄, H₂PO₄, 0.5CO₃, 0.5SO₄, ⅓PO₄, CH₃SO₃, CF₃SO₃, NTf₂, BF₄, acetate, tosylate, benzoate, salicylate, succinate, saccharinate, —OH and gluconate;
   n is an integer in a range from 1 to 36; and
   m is an integer in a range from 1 to 36.

The term “alkyl” or “alkyl groups” in the context of the present invention and as used herein refers to saturated hydrocarbons having one or more carbon atoms, including straight chain monovalent alkyl groups (e.g. methyl, ethyl, propyl, butyl, pentyl, hexyl, heptyl, octyl, nonyl, decyl, etc.), branched chain monovalent alkyl groups (e.g. isopropyl, tert-butyl, sec-butyl, isobutyl, etc.) or cyclic alkyl groups (cycloalkyl) (e.g. cyclopropyl, cyclopentyl, cyclohexyl, cycloheptyl, cyclooctyl, etc.). In a preferred variant the alkyl group is a straight chain or a branched chain alkyl group.

Unless otherwise specified, the term “alkyl” includes both unsubstituted or substituted alkyl. The term “substituted alkyls” refers to alkyl groups having substituents replacing one or more hydrogens on one or more carbons of the hydrocarbon backbone. Such substituents may include, for example, alkenyl, alkynyl, halogeno, hydroxyl, alkylcarbonyloxy, arylcarbonyloxy, alkyoxycrabonyloxy, aryloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkyoxyl, phosphate, phosphonato, phospinato, cyano, amino (including alkyl amino, dialkylamino, arylamino, diarylamino and alkylarylamino), acylamio (including alkylcarbonylamino, arylcarbonylamino, carbamoyl and ureido), imino, sulfydryl, alkylthio, arylthiol, thiocarboxylate, sulfates, alkylsulfinyl, sulfonates, sulfamoyl, sulfonamido, nitro, trifluormethyl, cyano, azido, heterocylic, alkylaryl or aromatic (including heteroaromatic groups).

In some variants, substituted alkyls can include a heterocyclic group. As used herein, the term “heterocyclic group” includes closed ring structures analogous to carbocyclic groups in which one or more of the carbon atoms in the ring is an element other than carbon, for example, nitrogen, sulfur or oxygen. Heterocyclic groups may be saturated or unsaturated. Exemplary heterocyclic groups include, but are not limited to, aziridine, ethylene oxide (epoxides, oxiranes), thiirane (episulfides), dioxirane, azetidine, oxetane, thietane, dioxetane, dithietane, dithiete, azolidine, pyrrolidine, pyrroline, oxolane, dihydrofuran, and furan.

Preferably, the alkyl groups according to the present invention are unsubstituted.

Alkyl groups generally includes those with 1 to 36 carbon atoms. Preferred examples of “alkyl” as used herein include C1 to C36 alkyl chains, i.e. the alkyl is selected from the group consisting of C1 alkyl, C2 alkyl, C3 alkyl, C4 alkyl, C5 alkyl, C6 alkyl, C7 alkyl, C8 alkyl, C9 alkyl, C10 alkyl, C10 alkyl, C11 alkyl, C12 alkyl, C13 alkyl, C14 alkyl, C15 alkyl, C16 alkyl, C17 alkyl, C18 alkyl, C19 alkyl, C20 alkyl. C21 alkyl, C22 alkyl, C23 alkyl. C24 alkyl, C25 alkyl, C26 alkyl, C27 alkyl, C28 alkyl, C29 alkyl, C30 alkyl, C31 alkyl, C32 alkyl, C33 alkyl, C34 alkyl, C35 alkyl and C36 alkyl.

In addition, the term “alkyl” may include “alkenylene”. The term “alkenylene” refers to a straight chain, branched or cyclic alkyl group having one or more carbon-carbon double bonds and further including at least one double bond. Alkenylene groups generally include those with 1 to 36 carbon atoms. The alkylene groups may be unsubstituted or substituted with those substituents similarly as described for the alkyl groups.

As used herein, “aryl” or “aromatic” groups are cyclic aromatic hydrocarbons that do not contains heteroatoms. Aryl groups include monocyclic, bicyclic, and poly cyclic ring systems. Thus, aryl groups include, but are not limited to, phenyl, azulenyl, heptalenyl, biphenylenyl, indacenyl, florenyl, phenanthrenyl, triphenylenyl, pyrenyl, naphthacenyl, chrysenyl, biphenyl, anthracenyl, indenyl, indanyl, pentalenyl, and naphthyl groups. In some embodiments, aryl groups contain 6 to14 carbons, in others from 6 to 12 or 6 to 10 carbon atoms in the ring portions of the groups. The phrase “aryl groups” includes groups containing fused rings, such as fused aromatic-aliphatic ring systems. Aryl groups may be unsubstituted or substituted with those substituents similarly as described for the alkyl groups.

In a preferred variant according to the first aspect of the present invention in general formula (II)
R₁ and R₂ are independently from each other selected from the group consisting of nitrogen and phosphor; and/or
R₃ and R₄ are independently from each other selected from the group consisting of H—, methyl, ethyl, n-propyl, i-propyl, n-butyl and sec-butyl; and/or
R₅ and R₆ are independently from each other selected from the group consisting of H—, methyl, ethyl, n-propyl, i-propyl n-butyl and sec-butyl;
R₇ is a C1 to C36 n-alkyl; and/or
X is selected from the group consisting of Cl, Br, F, I, HSO₄, 0.5HPO₄, H₂PO₄, 0.5CO₃, 0.5SO₄, ⅓PO₄, CH₃SO₃, CF₃SO₃, NTf₂, BF₄, acetate, tosylate, benzoate, salicylate, succinate, saccharinate, —OH and gluconate;
R₉, R₁₀ and R₁₁ are independently from each other selected from the group consisting of H—, methyl, ethyl, n-propyl and n-butyl; and/or
n is an integer from 1 to 36; and/or
m is an integer from 1 to 36.

In a more preferred variant according to the first aspect of the present invention, in general formula (II)
R₁ and R₂ are nitrogen; and/or
R₃ and R₄ are independently from each other selected from the group consisting of methyl, ethyl, n-propyl, and n-butyl; and/or
R₅ and R₆ are independently from each other selected from the group consisting of methyl, ethyl, n-propyl, and n-butyl; and/or
R₇ is a C6 to C24 n-alkyl; and/or
X is selected from the group consisting of Cl, Br, F and I; and/or
R₉, R₁₀ and R₁₁ are independently from each other selected from the group consisting of H—, methyl, ethyl, and n-propyl; and/or
n is an integer from 10 to 20; and/or
m is an integer from 10 to 20;

In a most preferred variant according to the first aspect of the present invention in general formula (II)
R₁ and R₂ are nitrogen; and/or
R₃ and R₄ are independently from each other selected from the group consisting of methyl and ethyl; and/or
R₅ and R₆ are independently from each other selected from the group consisting of methyl and ethyl; and/or
R₇ is a C10 to C20 n-alkyl; and/or
X is selected from the group consisting of Cl and Br; and/or
R₉, R₁₀ and R₁₁ are independently from each other selected from the group consisting of H— and ethyl; and/or
n is an integer from 12 to 18 and/or
m is an integer from 12 to 18.

Among the above specified compounds (a) the quaternary silyl compounds represented by the general formula (II) are particularly advantageous, since they form a long-lasting film upon application.

In a further preferred variant according to the first aspect of the present invention, the composition comprises at least two quaternary compounds according to the general formula (I) and/or general formula (II). This means, the composition comprises a mixture of two or more quaternary compounds according to the general formula (I) and/or general formula (II).

In a still more preferred variant according to the first aspect of the present invention, the composition comprises a mixture of at least four quaternary compounds according to the general formula (I) and/or general formula (II). Suchlike compositions exhibit an enhanced antimicrobial efficacy against a broad spectrum of microorganism.

In a most preferred variant according to the first aspect of the present invention, the quaternary compound according to the general formulae (I) or (II) is selected from the group consisting of N1-hexadecyl-N1,N1,N2,N2-tetramethyl-ethane-1-diaminium chloride, N1-octadecyl-N1,N1,N2,N2-tetramethyl-ethane-1-diaminiumchloride, N1-hexadecyl-N1,N1,N2,N2-tetramethyl-N2-(3-(trihydroxysilyl-)propyl-)ethane-1,2-diaminium chloride, N1-octacecyl-N1,N1,N2,N2-tetramethyl-N2-(3-(trihydroxysilyl-)propyl-)ethane-1,2-diaminium chloride, and any mixture thereof.

In a still more preferred variant according to the first aspect of the present invention, the composition comprises a mixture of at least two quaternary compounds according to the general formula (I) or (II), namely a combination selected from the group consisting of
N1-hexadecyl-N1,N1,N2,N2-tetramethyl-ethane-1-diaminium chloride and N1-octadecyl-N1,N1,N2,N2-tetramethyl-ethane-1-diaminiumchloride;
N1-hexadecyl-N1,N1,N2,N2-tetramethyl-ethane-1-diaminium chloride and N1-hexadecyl-N1,N1,N2,N2-tetramethyl-N2-(3-(trihy-droxysilyl-)propyl-)ethane-1,2-diaminium chloride;
N1-hexadecyl-N1,N1,N2,N2-tetramethyl-ethane-1-diaminium chloride and N1-octa-cecyl-N1,N1,N2,N2-tetramethyl-N2-(3-(trihydroxysilyl-)propyl-)ethane-1,2-diaminium chloride;
N1-octadecyl-N1,N1,N2,N2-tetramethyl-ethane-1-diaminiumchloride and N1-hexadecyl-N1,N1,N2,N2-tetramethyl-N2-(3-(trihydroxysilyl-)propyl-)ethane-1,2-diaminium chloride;
N1-octadecyl-N1,N1,N2,N2-tetramethyl-ethane-1-diaminiumchloride and N1-octacecyl-N1,N1,N2,N2-tetramethyl-N2-(3-(trihydroxysilyl-)propyl-)ethane-1,2-diaminium chloride; and
N1-hexadecyl-N1,N1,N2,N2-tetramethyl-N2-(3-(trihydroxysilyl-)propyl-)ethane-1,2-diaminium chloride and N1-octacecyl-N1,N1,N2,N2-tetramethyl-N2-(3-(trihydroxysilyl-)propyl-)ethane-1,2-diaminium chloride;
N1-hexadecyl-N1,N1,N2,N2-tetramethyl-ethane-1-diaminium chloride and N1-octadecyl-N1,N1,N2,N2-tetramethyl-ethane-1-diaminiumchloride and N1-hexadecyl-N1,N1,N2,N2-tetramethyl-N2-(3-(trihydroxysilyl-)propyl-)ethane-1,2-diaminium chloride;
N1-hexadecyl-N1,N1,N2,N2-tetramethyl-ethane-1-diaminium chloride and N1-hexadecyl-N1,N1,N2,N2-tetramethyl-N2-(3-(trihydroxysilyl-)propyl-)ethane-1,2-diaminium chloride and N1-octacecyl-N1,N1,N2,N2-tetramethyl-N2-(3-(trihydroxysilyl-)propyl-)ethane-1,2-diaminium chloride; and
N1-octadecyl-N1,N1,N2,N2-tetramethyl-ethane-1-diaminiumchloride and N1-hexadecyl-N1,N1,N2,N2-tetramethyl-N2-(3-(trhydroxysilyl-)propyl-)ethane-1,2-diaminium chloride and N1-octacecyl-N1,N1,N2,N2-tetramethyl-N2-(3-(trihydroxysilyl-)propyl-)ethane-1,2-diaminium chloride.

In a most preferred variant according to the first aspect of the present invention, the composition comprises a mixture of N1-hexadecyl-N1,N1,N2,N2-tetramethyl-ethane-1-diaminium chloride, N1-octadecyl-N1,N1,N2,N2-tetramethyl-ethane-1-diaminiumchloride, N1-hexadecyl-N1,N1,N2,N2-tetramethyl-N2-(3-(trihydroxysilyl-)propyl-)ethane-1,2-diaminium chloride and N1-octacecyl-N1,N1,N2,N2-tetramethyl-N2-(3-(trihydroxysilyl-)propyl-)ethane-1,2-diaminium chloride.

The above compounds according to general formula (I) and (II) are known as substances with antimicrobial efficacy.

An antimicrobial is an agent that kills microorganisms or stops their growth. Antimicrobial medicines can be grouped according to the microorganisms they act primarily against. In the context of the present invention, the term antimicrobial or antimicrobial substance/agent includes antibacterials, antivirals, antifungals, and antiparasitics.

However, it turned out that the above single compounds according to general formula (I) and (II) as such or any mixture thereof do not exhibit activity or not sufficient yeasticidal and fungicidal activity in the tested concentrations of 10%, 50% and 80% at a contact time of 5 minutes against the tested strains C. albicans and A. brasiliensis (according to DIN EN 1650).

The quaternary compound (a) is present in the composition according to the present invention in an amount of 0.001 to 2 wt.-%, preferably in an amount of 0.01 to 1.5 wt.-% and still more preferred in an amount of 0.1 to 0.5 wt.-%, based on the total weight of the composition.

For a mixture of compounds (a), the above amounts relate to the total content of the compounds (a) in the mixture, i.e., the amount is the sum of the content of the single compounds (a) in the mixture.

The component (b) of the composition according to the first aspect of the present invention relates to at least one alcohol or polyol.

The alcohol and/or polyol component (b) enhances the permeability of the cell membrane of the microorganism to be killed. With the enhancement of the cell membrane permeability, the antimicrobial agents of the composition can better permeate into the microorganism cells.

The at least one alcohol and/or polyol is selected from the group consisting of ethanol, n-propanol, 2-propanol, n-butanol, 2-butanol, 3-butanol, tert-butanol, 2-methyl-1-butanol, 3-methyl-1-butanol, 3-methyl-2-butanol, 3-methoxy-3-methyl-1-butanol, 2-methyl-2-butanol, 3,3-dimethylbutane-2-ol, 2,2-dimethyl-1-propanol, hexane-1-ol, hexane-2-ol, hexane-3-ol, 2-methylpentane-1-ol, 2-methylpentane-2-ol, 2-methylpentane-3-ol, 4-methylpentane-1-ol, 4-methylpentane-2-ol, 3-methylpentane-1-ol, 3-methylpentane-2-ol, 3-methylpentane-3-ol, 2,2-dimethylbutane-1-ol, 3,3-dimethylbutane-1-ol, 3,3-dimethylbutane-2-ol, 2,3-dimethylbutane-2-ol, 2,3-dimethylbutane-1-ol, 2-ethylbutane-1-ol, glycerol, propylene glycol, butylene glycol, pentylene glycol, hexylene glycol, pentaerythritol, trimethylolpropane, 1,2-ethandiol, 1,4,6-octanetriol, propanediol, butanediol, pentanediol, hexanediol, octanediol, chloropentanediol, glycerol monoalkyl ether, glycerol monoethyl ether, diethylene glycol, dipropylene glycol, 2-ethylhexanediol-1,4, cyclohexanediol-1,4, 1,2,6-hexanetriol, 1,3,5-hexanetriol, 1,2,3-hexanetriol, 1,3-bis-(2-hydroxyethoxy)propane, heptane-2,4,6-triol, heptane-1,2,3-triol, heptane-1,4,7-triol, heptane-1,2,7-triol, neopentyl glycol, 2-methyl propanediol, trimethylol propane monoallylether, 2,2,4-timethyl-1,3-pentanediol, 1,4-butanediol, cyclohexanedimethanol, and 2,2-dimethyl-3-hydroxypropyl-2,2-dimethyl-3-hydroxypropionate, phenoxyethanol, hexylresorcinol, phenethylalcohol, benzylalcohol and any mixture thereof.

Particular beneficial are composition according to the present invention, wherein the at least one alcohol and/or polyol component (b) is selected from the group consisting of ethanol, n-propanol, 2-propanol, n-butanol, 2-butanol, 3-butanol, tert.-butanol, 2-methyl-1-butanol, 3-methyl-1-butanol, 3-methyl-2-butanol, 3-methoxy-3-methyl-1-butanol, 2-methyl-2-butanol, 3,3-dimethylbutan-2-ol, 2,2-dimethyl-1-propanol, 2-methylpentane-2-ol, 4-methylpentane-2-ol, 3-methylpentane-2-ol, 3-methylpentane-3-ol, 2,2-dimethylbutane-1-ol, 3,3-dimethylbutane-2-ol, 2,3-dimethylbutane-2-ol, 2,3-dimethylbutane-1-ol, 2-ethylbutane-1-ol, glycerol, propylene glycol, butylene glycol, pentylene glycol, hexylene glycol, pentaerythritol, trimethylolpropane, 1,2-ethandiol, propanediol, butanediol, pentanediol, hexanediol, glycerol monoalkyl ether, glycerol monoethyl ether, diethylene glycol, dipropylene glycol, 2-ethylhexanediol-1,4, 1,2,6-hexanetriol, 1,3,5-hexanetriol, 1,2,3-Hexanetriol, heptane-2,4,6-triol, heptane-1,2,3-triol, heptane-1,4,7-triol, heptane-1,2,7-triol, neopentyl glycol, 2-methyl propanediol, trimethylol propane monoallylether, 1,4-butanediol, hexylglycerin, heptylglycerin, and any mixture thereof.

In a preferred variant, the at least one alcohol and/or polyol component (b) is selected from the group consisting of ethanol, n-propanol, 2-propanol, n-butanol, 2-butanol, 3-butanol, tert.-butanol, 2-methyl-1-butanol, 3-methyl-1-butanol, 3-methyl-2-butanol, 3-methoxy-3-methyl-1-butanol, 2-methyl-2-butanol, 3,3-dimethylbutan-2-ol, 2,2-dimethyl-1-propanol, 2-ethylbutan-1-ol, glycerol, propylene glycol, butylene glycol, pentylene glycol, hexylene glycol, pentaerythritol, trimethylolpropane, 1,2-ethandiol, propanediol, butanediol, pentanediol, hexanediol, glycerol monoalkyl ether, glycerol monoethyl ether, diethylene glycol, dipropylene glycol, 2-ethylhexanediol-1,4, neopentyl glycol, 2-methyl propanediol, 1,4-butanediol, and any mixture thereof.

In a still more preferred variant, the at least one alcohol and/or polyol component (b) is selected from the group consisting of ethanol, n-propanol, 2-propanol, n-butanol, 2-butanol, 3-butanol, tert.-butanol, 2-methyl-1-butanol, 3-methyl-1-butanol, 3-methyl-2-butanol, 3-methoxy-3-methyl-1-butanol, glycerol, propylene glycol, butylene glycol, pentylene glycol, hexylene glycol, pentaerythritol, trimethylolpropane, 1,2-propanediol, butanediol, pentanediol, hexanediol, diethylene glycol, dipropylene glycol, 2-methyl propanediol, 1,4-butanediol, and any mixture thereof.

In a most preferred variant, the at least one polyol component is pentylene glycol.

The alcohol and/or polyol component (b) is present in the composition according to the present invention in an amount of 0.0001 to 15 wt.-%, preferably in an amount of 0.0001 to 10 wt.-% and still more preferred in an amount of 0.0001 to 8 wt.-%, based on the total weight of the composition.

For an alcohol and/or polyol mixture, the above amounts relate to the total content of the alcohols and/or polyols in the mixture, i.e., the amount is the sum of the content of the single alcohols and/or polyols in the mixture.

The component (c) of the composition according to the first aspect of the present invention relates to at least one inorganic and/or organic acid or a salt or a derivative thereof.

The inorganic or organic acid in the composition according to the present invention affects the metabolism of the microorganism to be killed.

The at least one inorganic and/or organic acid component (c) is selected from the group consisting of salicylic acid, sorbic acid, lactic acid, hydrochloric acid, boric acid, sulfuric acid, hypo sulfuric acid, phosphoric acid, perchloric acid, peroxymonosulfuric acid, peroxydisulfuric acid, hypodiphosphonic acid, hypodiphosphoric acid, diphosphonic acid, diphosphonc acid, benzoic acid, anisic acid, formic acid, butanoic acid, propionic acid, citric acid, acetic acid, acetylsalicylic acid, ascorbic acid, malic acid, tartaric acid, gallic acid, glyoxylic acid, fumaric acid, glycolic acid, ferulic acid, mandelic acid, acrylic acid, malonic acid, hypophosphorous acid/phosphinic acid, sulfurous acid, peroxodiphosphoric acid, artemisinic acid, peroxophosphoric acid, permanganic acid, undecylenic acid, levulinic acid, 1-naphthylacetic acid, hydroxamic acid, caprylic acid, caproic acid, adipic acid, gamma-hydroxybutyric acid, capric acid, ellagic acid, usnic acid, ricinoleic acid, quinic acid, chorismic acid, isophthalic acid, oxaloacetic acid, squaric acid, oleic acid, bicinchoninic acid, barbituric acid, citronellic acid, crotonic acid, isocrotonic acid, vinyl acetic acid, succinic acid, shikimic acid, maleic acid, valeric acid, clavulanic acid, alendronic acid, arachic acid, arachidonic acid, behenic acid, hydrocyanic acid, pyruvic acid, carbamic acid, linoleic acid, alpha-linolenic acid, cerotic acid, fluor acetic acid, chloro acetic acid, bromo acetic acid, lodo acetic acid, peracetic acid, glutamic acid, aspartic acid, methanesulfonic acid, erucic acid, fusaric acid, fusidic acid, gamma-aminobutyric acid, gondosoic acid, glucaric acid, gluconic acid, lauric acid, lignoceric acid, margaric acid, melissic acid, montanic acid, myristic acid, nermonic acid, oxaacetic acid, palmitoleic acid, linseedic acid, thiosulfuric acid, trichloroacetic acid, trifluoromethanesulfuric acid, trifluoroacetic acid, abietic acid, cinnamic acid, vulpic acid, tetrahydrofolic acid, phthalic acid, uric acid, fusidic acid, phenylacetic acid, N-acetlyneuramic acid, indole-3-acetic acid, ibotenic acid, hippuric acid, folic acid, terephthalic acid, sulphanilic acid, styphnic acid, picric acid, heptanoic acid, pimelic acid, suberic acid, azelaic acid, sebacic acid, pelargonic acid, isocitric acid, aconitic acid, allylacetic acid, glutaric acid, alpha-ketoglutaric acid, 2,5-dihydroxybenzoic acid, itaconic acid, perillaic acid, 3,4-dihydroxybenzoic acid, capryloyl glycine, PEG-6 undecylenate, propane caprylate, undecylenoyl glycine, undecylenoyl sarcosine, zink glycinate salicylate, amino-levulinic acid-hydrochlorid, biphenyl hydroxamic acid, C12-C15 pareth-3-benzoate, capryloyl methyl serinate, capryloyl serinate, capryloyl salicylic acid, caprylyl glycol linseedate, ricinoleic acid benzoate, cetyl ricinoleic acid benzoate, sorbityl dimonium anisate (CAS-No.: 2097714-04-8, zink cysteiante, any mixture thereof, and salts and derivatives of the afore-mentioned acids.

Particular beneficial are composition according to the present invention, wherein the at least one inorganic and/or organic acid component (c) is selected from the group consisting of salicylic acid, sorbic acid, lactic acid, hydrochloric acid, boric acid, sulfuric acid, hypo sulfuric acid, phosphoric acid, perchloric acid, peroxymonosulfuric acid, hypodiphosphonic acid, hypodiphosphoric acid, diphosphonic acid, diphosphoric acid, benzoic acid, anisic acid, formic acid, butanoic acid, propionic acid, citric acid, acetic acid, acetylsalicylic acid, ascorbic acid, malic acid, tartaric acid, gallic acid, glyoxylic acid, fumaric acid, glycolic acid, ferulic acid, mandelic acid, acrylic acid, malonic acid, hypophosphorous acid/phosphinic acid, sulfurous acid, peroxodiphosphoric acid, peroxophosphoric acid, undecylenic acid, levulinic acid, hydroxamic acid, caprylic acid, caproic acid, adipic acid, capric acid, oxaloacetic acid, citronellic acid, crotonic acid, isocrotonic acid, vinyl acetic acid, shikimic acid, maleic acid, valeric acid, pyruvic acid, carbamic acid, fluor acetic acid, chloro acetic acid, bromo acetic acid, iodo acetic acid, peracetic acid, glutamic acid, aspartic acid, methanesulfonic acid, erucic acid, glucaric acid, gluconic acid, thiosulfuric acid, trichloroacetic acid, trifluoromethanesulfuric acid, trifluoroacetic acid, cinnamic acid, uric acid, allylacetic acid, glutaric acid, 2,5-dihydroxybenzoic acid, 3,4-dihydroxybenzoic acid, capryloyl glycine, PEG-6 undecylenate, propane caprylate, undecylenoyl glycine, undecylenoyl sarcosine, zink glycinate salicylate, amino-levulinic acid-hydrochlorid, biphenyl hydroxamic acid, capringlycerin, caprylylglycerin, any mixture thereof, and salts and derivatives of the afore-mentioned acids.

In a preferred variant, the at least one inorganic and/or organic acid component (c) is selected from the group consisting of salicylic acid, sorbic acid, lactic acid, benzoic acid, anisic acid, citric acid, acetic acid, acetylsalicylic acid, ascorbic acid, malic acid, tartaric acid, gallic acid, glyoxylic acid, glycolic acid, ferulic acid, mandelic acid, levulinic acid, hydroxamic acid, caprylic acid, caproic acid, capric acid, oxaloacetic acid, citronellic acid, shikimic acid, maleic acid, pyruvic acid, carbamic acid, peracetic acid, glutamic acid, aspartic acid, glucaric acid, gluconic acid, oxaacetic acid, cinnamic acid, uric acid, glutaric acid, 2,5-dihydroxybenzoic acid, 3,4-dihydroxybenzoic acid, and any mixture thereof, and salts and derivatives of the afore-mentioned acids.

In a still more preferred variant, the at least one inorganic and/or organic acid component (c) is selected from the group consisting of salicylic acid, sorbic acid, lactic acid, benzoic acid, anisic acid, citric acid, acetylsalicylic acid, ferulic acid, mandelic acid, levulinic acid, hydroxamic acid, caprylic acid, caproic acid, capric acid, citronellic acid, shikimic acid, carbamic acid, cinnamic acid, 2,5-dihydroxybenzoic acid, 3,4-dihydroxybenzoic acid, and any mixture thereof, and salts and derivatives of the afore-mentioned acids.

In a most preferred variant, the at least one acid component (c) is benzoic acid and/or salicylic acid.

The salts of the above specified inorganic or organic acids are preferably selected from the group consisting of ammonium, alkali, earth alkali, aluminium, zinc, iron, copper, and silver salts.

Preferred derivates of the above specified inorganic or organic acids include their ether and ester derivates.

The inorganic or organic acid component (c) is present in the composition according to the present invention in an amount of 0.0001 to 4 wt.-%, preferably in an amount of 0.0001 to 3 wt.-% and still more preferred in an amount of 0.0001 to 2 wt.-%, based on the total weight of the composition. The afore-mentioned values relate to the stoichiometric acid portion.

For an acid mixture, the above amounts relate to the total content of the acids in the mixture, i.e., the amount is the sum of the content of the single acids in the mixture.

The component (d) of the composition according to the first aspect of the present invention relates to at least one essential oil which is distilled or extracted from various plants.

The essential oil in the composition according to the present invention has a proven antimicrobial effect.

The at least one essential oil component (d) is selected from the group consisting of Mentha piperita (peppermint) oil (CAS-No: 8006-90-4), Mentha spicata oil, Salvia sclarea oil, Fragaria ananassa seed oil, Rosmarinus officinalis oil (CAS-No: 8000-25-7), Origanum oil (CAS-No.: 8007-11-2), Achillea millefolium oil, Nageia nagi oil, Peumus boldus oil, Salvia officinalis oil (CAS-No: 8022-56-8), Sorbus aucuparia seed oil, Wrightia tinctoria oi, Abies alba oil, Abies pectinata oil, Adenosma nelsonioides oil, aeollanthus, Bakuchiol suaveolens oil, Aframomum angustifolium oil, Aleurites moluccanus bakoly oil, alligator oil, Allium cepa bulb oil, Artemisia absinthium oil, Bakuchiol, Calophyllum inophyllum oil, Cannabis sativa oil, Camellia sinensis oil, Chamaecyparis formosensis oil, Cinnamomum cassia bark oil, Cinnamosma fragrans oil, Clausena lansium oil, Commiphora gileadensis oil, Cyperus scariosus rhizome oil, Dendropanax melaleuca alternifolia oil (CAS-No: 68647-73-4), Morbiferus stem sap oil, Dracocephalum foetidum oil, Dracocephalum moldavica oil, Eugenia uniflora oil, Ferula galbaniflua resin oil, Hippophae rhamnoides oil, hydrogenated Nepeta cataria oil, Leptospermum laevigatum oil, Lindera umbellata oil, Lippia origanoides oil, Mentha suaveolens oil, Pistacia lenticus gum oil (CAS-No: 61789-92-2), Pistacia lenticus leaf oil (CAS-No: 90082-82-9), Psiadia altissima leaf oil, Salvia triloba oil, Sapindus mukorossi oil, Schinus terebinthifolia oil, Schizochytrium limacinum oil, Tagetes lemmonii oil, Thujopsis dolabrata oil, Thymus vulgaris oil (CAS-No: 84929-51-1), Torreya nucifera leaf oil (CAS-No: 84929-51-1), Ocimum basilicum oil (CAS-No.: 84775-71-3), Pistacia lenticus gum, and any mixture thereof.

Particular beneficial are compositions according to the present invention, wherein the at least one essential oil component (d) is selected from the group consisting of Rosmarinus officinalis oil (CAS-No: 8000-25-7), Origanum oil (CAS-No.: 8007-11-2), Salvia officinalis oil (CAS-No: 8022-56-8), Bakuchiol Suaveolens oil, Cinnamomum cassia bark oil, Cinnamosma fragrans oil, Pistacia lenticus gum oil (CAS-No: 61789-92-2), Pistacia lenticus leaf oil (CAS-No: 90082-82-9), Psiadia altissima leaf oil, Salvia triloba leaf oil, alligator oil, Artemisia absinthium oil, Melaleuca alternifolia oil (CAS-No.: 68647-73-4), Mentha piperita (peppermint) oil, Mentha spicata oil, Salvia sclarea oil, Ferula galbaniflua resin oil, Cinnamosma fragrans oil, Mentha suaveolens oil, Pistacia lenticus gum, Salvia triloba oil, Schinus terebinthifolia oil, Thymus vulgaris oil (CAS-No.: 84929-51-1), Ocimum basilicum oil (CAS-Nr. 84775-71-3), and any mixture thereof.

In a still more preferred variant, the at least one essential oil component (d) is selected from the group consisting of Rosmarinus officinalis oil (CAS-No: 8000-25-7), Origanum oil (CAS-No.: 8007-11-2), Salvia officinalis oil (CAS-No: 8022-56-8), Bakuchiol suaveolens oil, Cinnamomum cassia bark oil, Pistacia lenticus gum oil (CAS-No: 61789-92-2), Pistacia lenticus leaf oil (CAS-No: 90082-82-9), Artemisia absinthium oil, Melaleuca alternifolia oil (CAS-No.: 68647-73-4), Mentha piperita (peppermint) oil, Mentha spicata oil, Mentha suaveolens oil, Pistacia lenticus gum, Salvia triloba oil, Thymus vulgaris oil (CAS-No.: 84929-51-1), Ocimum basilicum oil (CAS-Nr. 84775-71-3), and any mixture thereof.

In a most preferred variant, the at least one essential oil is Pistacia lenticus gum oil (CAS-No: 61789-92-2) and/or Pistacia lenticus leaf oil (CAS-No: 90082-82-9).

The essential oil component (d) is present in the composition according to the present invention in an amount of 0.0001 to 5 wt.-%, preferably in an amount of 0.0001 to 1 wt.-% and still more preferred in an amount of 0.0001 to 0.3 wt.-%, based on the total weight of the composition.

For a mixture of essential oils, the above amounts relate to the total content of the essential oils in the mixture, i.e., the amount is the sum of the content of the single essential oils in the mixture.

In a preferred variant the composition according to the first aspect of the present invention comprises or consists of
(a) at least one quaternary compound selected from the group consisting of N1-hexadecyl-N1,N1,N2,N2-tetramethyl-ethane-1-diaminium chloride, N1-octadecyl-N1,N1,N2,N2-tetramethyl-ethane-1-diaminiumchloride, N1-hexadecyl-N1,N1,N2,N2-tetramethyl-N2-(3-(trihydroxysilyl-)propyl-)ethane-1,2-diaminium chloride and N1-octacecyl-N1,N1,N2,N2-tetramethyl-N2-(3-(trihydroxysilyl-)propyl-)ethane-1,2-diaminium chloride;
(b) at least one alcohol and/or polyol selected from the group consisting of ethanol, n-propanol, 2-propanol, n-butanol, 2-butanol, 3′-butanol, tert.-butanol, 2-methyl-1-butanol, 3-methyl-1-butanol, 3-methyl-2-butanol, 3-methoxy-3-methyl-1-butanol, glycerol, propyleneglycol, butyleneglycol, pentylene glycol, hexyleneglycol, pentaerythritol, trimethylolpropane, 1,2-propanediol, butanediol, pentanediol, hexanediol, diethylene glycol, dipropylene glycol, 2-methyl propanediol, 1,4-butanediol, and any mixture thereof;
(c) at least one inorganic and/or organic acid selected from the group consisting of salicylic acid, sorbic acid, lactic acid, benzoic acid, anisic acid, citric acid, acetylsalicylic acid, ferulic acid, mandelic acid, levulinic acid, hydroxamic acid, caprylic acid, caproic acid, capric acid, citronellic acid, shikimic acid, carbamic acid, cinnamic acid, 2,5-dihydroxybenzoic acid, 3,4-dihydroxybenzoic acid, and any mixture thereof, and salts and derivatives of the afore-mentioned acids as defined above; and
(d) at least one essential oil selected from the group consisting of Rosmarinus officinalis oil (CAS-No: 8000-25-7), Origanum oil (CAS-No.: 8007-11-2), Salvia officinalis oil (CAS-No: 8022-56-8), Bakuchiol suaveolens oil, Cinnamomum cassia bark oil, Pistacia lenticus gum oil (CAS-No: 61789-92-2), Pistacia lenticus leaf oil (CAS-No: 90082-82-9), Artemisia absinthium oil, Melaleuca alternifolia oil (CAS-No.: 68647-73-4), Mentha piperita (peppermint) oil, Mentha spicata oil, Mentha suaveolens oil, Pistacia lenticus gum, Salvia triloba oil, Thymus vulgaris oil (CAS-No.: 84929-51-1), Ocimum basilicum oil (CAS-Nr. 84775-71-3), and any mixture thereof.

In a more preferred variant, the composition according to the present invention comprises or consists of
(a) a mixture of N1-hexadecyl-N1,N1,N2,N2-tetramethyl-ethane-1-diaminium chloride, N1-octadecyl-N1,N1,N2,N2-tetramethyl-ethane-1-diaminiumchloride, N1-hexadecyl-N1,N1,N2,N2-tetramethyl-N2-(3-(trihydroxysilyl-)propyl-)ethane-1,2-diaminium chloride and N1-octacecyl-N1,N1,N2,N2-tetramethyl-N2-(3-(trihydroxysilyl-)propyl-)ethane-1,2-diaminium chloride;
(b) at least one alcohol and/or polyol selected from the group consisting of ethanol, n-propanol, 2-propanol, n-butanol, 2-butanol, 3′-butanol, tert.-butanol, 2-methyl-1-butanol, 3-methyl-1-butanol, 3-methyl-2-butanol, 3-methoxy-3-methyl-1-butanol, glycerol, propylene glycol, butylene glycol, pentylene glycol, hexylene glycol, pentaerythritol, trimethylolpropane, 1,2-propanediol, butanediol, pentanediol, hexanediol, diethylene glycol, dipropylene glycol, 2-methyl propanediol, 1,4-butanediol, and any mixture thereof;
(c) at least one inorganic and/or organic acid selected from the group consisting of salicylic acid, sorbic acid, lactic acid, benzoic acid, anisic acid, citric acid, acetylsalicylic acid, ferulic acid, mandelic acid, levulinic acid, hydroxamic acid, caprylic acid, caproic acid, capric acid, citronellic acid, shikimic acid, carbamic acid, cinnamic acid, 2,5-dihydroxybenzoic acid, 3,4-dihydroxybenzoic acid, and any mixture thereof, and salts and derivatives of the afore-mentioned acids as defined above; and
(d) at least one essential oil selected from the group consisting of Rosmarinus officinalis oil (CAS-No: 8000-25-7), Origanum oil (CAS-No.: 8007-11-2), Salvia officinalis oil (CAS-No: 8022-56-8), Bakuchiol suaveolens oil, Cinnamomum cassia bark oil, Pistacia lenticus gum oil (CAS-No: 61789-92-2), Pistacia lenticus leaf oil (CAS-No: 90082-82-9), Artemisia absinthium oil, Melaleuca alternifolia oil (CAS-No.: 68647-73-4), Mentha piperita (peppermint) oil, Mentha spicata oil, Mentha suaveolens oil, Pistacia lenticus gum, Salvia triloba oil, Thymus vulgaris oil (CAS-No.: 84929-51-1), Ocimum basilicum oil (CAS-Nr. 84775-71-3), and any mixture thereof.

In a most preferred variant, the composition according to the present invention comprises or consists of
(a) a mixture of N1-hexadecyl-N1,N1,N2,N2-tetramethyl-ethane-1-diaminium chloride, N1-octadecyl-N1,N1,N2,N2-tetramethyl-ethane-1-diaminiumchloride, N1-hexadecyl-N1,N1,N2,N2-tetramethyl-N2-(3-(trihydroxysilyl-)propyl-)ethane-1,2-diaminium chloride and N1-octacecyl-N1,N1,N2,N2-tetramethyl-N2-(3-(trihydroxysilyl-)propyl-)ethane-1,2-diaminium chloride;
(b) pentylene glycol,
(c) benzoic acid and/or salicylic acid; and
(d) Pistacia lenticus gum oil (CAS-No: 61789-92-2) and/or Pistacia lenticus leaf oil (CAS-No: 90082-82-9).

In a preferred variant the composition according to the present invention further encompasses
(e) at least one antimicrobial component which is different from the quaternary component (a); and/or
(f) at least one quaternary organic nitrogen or phosphorous containing component; and/or
(g) at least one ten side component.

In order to optimize or further enhance the antimicrobial efficacy, the composition is advantageously combined with at least one further antimicrobial, i.e. antibacterial, antiviral, antifungal or antiparasitic, agent, which is different from the quaternary compound (a) as defined above.

The further different antimicrobial agent may provide reliable protection against other microorganisms as described above. Hence, the combination with a further different antimicrobial agent allows antimicrobial protection against different groups of microorganisms, and, thus, a broader spectrum of microorganism.

The one or more further different antimicrobial agent is selected from the group comprising or consisting of benzethonium chloride, benzalkonium chloride, thymol, lactic acid, chitosan, triethylene glycol, diols, 1,2-hexanediol, octanoic acid, sodium dichloroisocyanurate, citric acid, caffeic acid, caffeic acid-Fe(III) chelate, quercetin-3-rutinoside (rutin), capsaicin, antimicrobial peptides, polylysine, polyethyleneimine, polyguanidines, phenolic or polyphenols, such as lignin and tannin, lycorine, chitosan, carbon nanotubes, fullerene, graphene, graphene oxide, peroxides, 13-caryophyllene, star anise oil, berberine chloride, palmatine, novobiocin, morin, polyamines, phenols, triclosan, dimethylamine, epichlorohydrin, superfloc C-572, quercetin, glycyrrhizic acid, sulfamic acid, glycolic acid, lauric acid, capric acid, or any mixture thereof, quaternary ammonium based compounds including, but are not limited to n-alkyl dimethylbenzyl ammonium chloride, di-n-octyl dimethylammonium chloride, dodecyl dimethyl ammonium chloride, n-alkyl dimethylbenzyl ammonium saccharinate, and 3-(trimethoxysilyl) propyldimethyloctadecyl ammonium chloride, poly diallyldimethylammonium chloride, poly DADMAC, polyhexamethylene biguanide (PHMB), chlorhexidine, Bardac 205M, 208M and BTC885, quaternary ammonium based silanes such as 3(trihydroxysilyl) propyldimethyloctadecyl ammonium chloride, or any mixture thereof, metal based such as metal salts, metal nano- and microparticles, and metallics compounds, including, but are not limited to zinc, zinc pyrithione, silver, silver nanoparticles, cerium, cerium oxide nanoparticles, selenium nanoparticles, copper nanoparticles, copper, gold, gold nanoparticles, titanium, titanium nanoparticles, iron, iron nanoparticles, platinum-, tin-, ruthenium-, cobalt-based antibacterial, antiviral, antifungal, and antiparasitic compounds, Caprylhydroxamic Acid, o-cymen-5-ol, Isopropylparaben, Capryloyl Glycine, Phenylpropanol, Tropolone, PCA Ethyl Cocoyl Arginate, 2-Methyl 5-Cyclohexylpentanol, Phenoxyethanol, Disodium EDTA, Methylparaben and its salts, Sodium Benzoate, Benzyl Alcohol, Potassium Sorbate, Benzyl Salicylate, Propylparaben, Methylchloroisothiazolinone, Methylisothiazolinone, Ethylhexylglycerin, Butylparaben, Ethylparaben, Sodium Propylparaben, DMDM Hydantoin, Hydroxyethoxyphenyl Butanone, Hydroxyethoxyphenyl Butanol, Itaconic Acid and salts thereof, Octopirox, Propanediol Caprylate, Climbazole, 4-Hydroxyacetophenone, Dehydroacetic Acid, Iodopropynyl Butylcarbamate, Salicylic Acid, Chlorphenesin, Isobutylparaben, Sodium Ethylparaben, Diazolidinyl Urea, Farnesol, Bisabolol, Glyceryl Caprylate, Sodium Phytate or Phytic acid, Sodium Levulinate or Levulinic Acid and esters thereof, Chlorhexidine, Glyceryl Laurate, Anisic Acid and salts thereof, Chlorhexidine Digluconate, TEA-Salicylate, Phenethyl Alcohol, Glyceryl Caprate, Sorbitan Caprylate, Tetrasodium Glutamate Diacetate, Trisodium Ethylenediamine Disuccinate, Sodium Lactate, Undecylenoyl glycine, Thymol, 1,3-Propanediol, 4-hydroxybenzoic acid and its salts and esters, N-(4-chlorophenyl)-N′-(3,4-dichlorophenyl)urea, 2,4,4′-trichloro-2′-hydroxy-diphenyl ether (triclosan), 4-chloro-3,5-dimethyl-phenol, 2,2′-methylenebis(6-bromo-4-chlorophenol), 3-methyl-4-(1-methylethyl)phenol, 2-benzyl-4-chloro-phenol, 3-(4-chlorophenoxy)-1,2-propanediol, 3-iodo-2-propynyl butylcarbamate, 3,4,4′-trichlorocarbanilide (TTC), antibacterial fragrances, thymol, thyme oil, eugenol, oil of cloves, menthol, mint oil, glycerol monocaprate, glycerol monocaprylate, glycerol monolaurate (GML), diglycerol monocaprate (DMC), salicylic acid N-alkylamides, such as, for example, n-octylsalicylamide or n-decylsalicylamide, Dehydroacetic Acid, 4-Methylbenzyl Alcohol, Frambinon, Xylityl caprylate, Benzoic acid 3-hyroxypropylester, Anisic acid 3-hydroxypropylester, Polyhexanid, Octenidine, 3-Hydroxypropyl 2-hydroxybenzoate and mixtures of two or more the aforesaid antimicrobial agents.

In a preferred variant, the one or more further antimicrobial active component is selected from the group comprising or consisting of Hydroxamic Acid, Caprylhydroxamic Acid, Isopropylparaben, Capryloyl Glycine, Phenylpropanol, Methylparaben and its salts, Sodium Benzoate, Benzyl Alcohol, Potassium Sorbate, Propylparaben, Methylchloroisothiazolinone, Methylisothiazolinone, Ethylhexylglycerin, Butylparaben, Ethylparaben, Sodium Propylparaben, DMDM Hydantoin, Octopirox, Propanediol Caprylate, Climbazole, Salicylic Acid, Chlorphenesin, Isobutylparaben, Sodium Ethylparaben, Diazolidines Urea, Glyceryl Caprylate, Phytic acid, Levulinic Acid, Chlorhexidine, Anisic Acid, Phenethyl Alcohol, Caprylic Acid, Lactic Acid, Undecylenic Acid, Propanediol, Triclosan, 3-iodo-2-propynyl butylcarbamate, Benzoic Acid, Polyhexanid, Octenidine and any mixture thereof.

The aforesaid antimicrobial component can be used either as a single component or in combination with one or more further antimicrobial components as specified above. To enhance the antimicrobial effect and to cover a broader range of antimicrobial effect, a combination of two or more antimicrobial components is preferably used.

The one or more antimicrobial component is comprised in the composition according to the present invention in an amount from 0.01 to 2.0 wt.-%, preferably in an amount from 0.01 to 1.0 wt.-%, more preferred in an amount from 0.01 to 0.5 wt.-%, based on the total weight of the composition.

In order to optimize or enhance the antimicrobial effect of the composition, the composition is further combined with at least one quaternary organic nitrogen or phosphorous containing component, which is different from the quaternary component (a) as defined above. They are cationic surfactants (positively charged surface-active agents) that impact cell walls and membranes after relatively long contact times. Their permanent positive charge makes them bind readily to the negatively charged surface of most microbes. The quaternary ammonium compounds are a family of antimicrobial compounds considered as potent in its disinfectant activity cationic actives, such as quaternary organic ammonium compounds with one or two cationically charged ammonium ions, as well as their inner salts with N-oxide molecular structure as well as quaternary organic phosphorus compounds with a maximum of two cationically charged phosphonium ions, for example poly-nitrogen compounds such as biguanidines (e.g. chlorhexidines, polyhexanides), bipyridines and polypyridines (e.g. octenidines). Such quaternary organic nitrogen or phosphorous containing components are commonly used in antimicrobial compositions and as such known.

In a preferred variant, the one or more further quaternary organic nitrogen or phosphorous containing component is selected from the group comprising or consisting of didecyldimethylammoniumchloride, lauramine oxide, cetearalkonium bromide, cetalkonium chloride, benzalkonium saccharinate, benzalkonium chloride, benzalkonium bromide, hydroxypropyl bistrimonium diiodide, benzalkonium cetyl phosphate, benzethonium chloride, tetrabutyl ammonium bromide, cetethyldimonium bromide, cetrimonium chloride, cetrimonium bromide, methylbenzethonium chloride, cetylpyridinium chloride, cetrimonium tosylate, cetrimonium saccharinate, cetrimonium methosulfate, chlorhexidine, chlorhexidine diacetate, chlorhexidine digluconate, chlorhexidine dihydrochloride, hexetidine, octenidine hydrochlorid, dequalinium chlorid, and any mixture thereof.

The aforesaid quaternary organic nitrogen or phosphorous containing component can be used either as a single component or in combination with one or more further quaternary organic components as specified above. In order to enhance the antimicrobial effect and to cover a broader range of antimicrobial effect, a combination of two or more of said quaternary organic components is preferably used.

The one or more further quaternary organic nitrogen or phosphorous containing component is comprised in the composition according to the present invention in an amount from 0.001 to 2.0 wt.-%, preferably in an amount from 0.005 to 1.0 wt.-%, more preferred in an amount from 0.01 to 0.5 wt.-%, based on the total weight of the composition.

To facilitate the processability, for example to improve the solubility of the single ingredients of the composition, to improve physical properties such as film building or to enhance foaming, at least one tenside or a mixture of tensides is added to the composition according to the present invention.

The tensides have solubilizing properties for the homogeneous incorporation of the otherwise water-insoluble essential oils in the final aqueous product. In addition, they provide a stable, sustainably non-evaporable solvation film for maintaining the solubility of the antimicrobially active components and ensure possible foaming of the final formulation by means of foam dispenser application.

The tenside may be a surfactant or a detergent or a mixture of surfactants or detergents, which is selected from the group of tensides consisting of nonionic, amphoteric, or cationic tensides. Preferably, the tenside is an amphoteric or a nonionic tenside.

The one or more tenside is selected from the group comprising or consisting of glyceryl trioctanoate, dibutyl sebacate, triethyl citrate, triacetin, myvacet (acetylated monoglycerides) and diethyl phthalate or 1,2-hexanediol, glycerol, caprylic/capric triglycerides, glycerol monolaurate, anionic detergent and surfactant such as sodium lauryl sulfate, alkylbenzene sulfonates, deoxycholic acid, sulfates include ammonium lauryl sulfate, (sodium dodecyl sulfate, sis, or sds), and the related alkyl-ether sulfates sodium laureth sulfate (sodium lauryl ether sulfate or sles), and sodium myreth sulfate, docusate (dioctyl sodium sulfosuccinate), perfluorooctanesulfonate (pfos), perfluorobutanesulfonate, alkyl-aryl ether phosphates, alkyl ether phosphates, carboxylates such as sodium stearate, sodium lauroyl sarcosinate, carboxylate-based fluorosurfactants such as perfluorononanoate, perfluorooctanoate (pfoa or pfo), or selected from the group comprising nonionic and zwitter ionic surfactants and detergens such as polysorbate 80, polyoxyethylene, glycoside, ethoxylates, fatty alcohol ethoxylates, narrow-range ethoxylate, octaethylene glycol monododecyl ether, pentaethylene glycol monododecyl ether, alkylphenol ethoxylates (apes or apeos), nonoxynols, triton x-100, fatty acid ethoxylates, special ethoxylated fatty esters and oils, ethoxylated amines and/or fatty acid amides, polyethoxylated tallow amine, cocamide monoethanolamine, cocamide diethanolamine, terminally blocked ethoxylates, poloxamers, fatty acid esters of polyhydroxy compounds, fatty acid esters of glycerol, glycerol monostearate, glycerol monolaurate, fatty acid esters of sorbitol, sorbitan monolaurate, sorbitan monostearate, sorbitan tristearate, tweens: tween 20, tween 40, tween 60, tween 80, fatty acid esters of sucrose, alkyl polyglucosides, alkyl polyglycoside such as decyl glucoside, lauryl glucoside, octyl glucoside, or cationinc detergents and surfactants, such as octenidine dihydrochloride, cetrimonium bromide (ctab), cetylpyridinium chloride (cpc), benzalkonium chloride (bac), benzethonium chloride (bzt), dimethyldioctadecylammonium chloride, and dioctadecyldimethylammonium bromide (dodab).

In a preferred variant, the one or more tenside is selected from the group comprising or consisting of glucoside tensides, sarcosinate tensides, sultaine tensides, betaine tensides, saponine tensides and any mixture thereof.

In a more preferred variant, the one or more tenside(s) is/are selected from the group consisting of cocamidoproypyl-betaine, betaine, arachidyl glucoside, glycinbetaine, palmitoyl glucoside, alkylglucoside, cocoglucoside and any mixture thereof.

The one or more tenside is comprised in the composition according to the present invention in an amount from 0.01 to 15.0 wt.-%, preferably in an amount from 0.05 to 10.0 wt.-%, more preferred in an amount from 0.1 to 5.0 wt.-%, based on the total weight of the composition.

The antimicrobial composition of the present invention can be either a liquid solution, a spray, or a film-forming formulation.

Thus, dependent on the application form, the composition according to the present invention can further comprise a solvent or a film forming material.

The solvent or a mixture of solvents is preferably selected from the group comprising or consisting of water, in particular demineralized water, a low molecular weight alcohol, alkylene glycol ether based solvents, including but are not limited to, ethylene glycol monopropyl ether, ethylene glycol monobutyl ether, ethylene glycol monohexyl ether, ethylene cyclo monohexyl ether, diethylene glycol monomethyl ether, diethylene glycol monoethyl ether, diethylene glycol monobutyl ether, diethylene glycol monohexyl ether, triethylene glycol monomethyl ether, triethylene glycol monoethyl ether, triethylene glycol monobutyl ether, propylene glycol methyl ether, propylene glycol methyl ether acetate, propylene glycol n-butyl ether, dipropylene glycol n-butyl ether, dipropylene glycol methyl ether, dipropylene glycol methyl ether acetate, propylene glycol n-propyl ether, dipropylene glycol n-propyl ether, and tripropylene glycol methyl ether, butanol, pentanol, acetone, isopropanol, ethyl acetate, glycerol, essential oils, pine oil, lemon oil, limonene, pinene, cymene, myrcene, fenchone, borneol, nopol, cineol, ionone, inorganic buffer, buffer solution, a terpene and terpene derivative, or any mixture thereof.

More preferred, the solvent is selected from the group comprising water, preferably demineralized water, a low molecular weight alcohol such as ethanol, or any mixture thereof.

Still more preferred, the solvent is selected from water, preferably demineralized water, pure ethanol, or denatured ethanol. In some aspects, the solvent is a mixture of ethanol and isopropanol.

Most preferred the solvent is a mixture of water, preferably demineralized water, and ethanol.

The amount of solvent in the composition is 5 to 99 wt.-%, preferably 5 to 95 wt.-%, more preferred 5 to 90 wt.-%, still more preferred 5 to 86 wt.-%, and most preferred 10 to 20 wt.-%.

To form a long-lasting antimicrobial and mechanical stable coating on the skin, on surfaces or on objects, the composition according to the present invention encompasses a film forming material.

The film forming material is preferably selected from the group comprising or consisting of ethyl cellulose, lignocellulose, cellulose derivative such as hydroxyethyl cellulose, cellulose acetate, nanocellulose, xanthan gum, gummi arabicum, carboxymethyl-cellulose, nanofibril cellulose, nanocrystalline cellulose, zein, polyethylene glycol, chitin, chitosan, polyvinyl alcohol, polylactic acid, polyglycolic acid, poly lactic-co-glycolic acid (PLGA), polycaprolactone, polysaccharide, polyvinyl alcohol, polyvinyl acetate, polyvinyl alcohol-polyethylene glycol graft-copolymer, acrylate polymers, poly(methacrylic acid)polymer or co-polymers, and any mixture thereof.

In a preferred variant, the film forming material is ethyl cellulose.

The composition comprises the film forming material in an amount from 0.01 to 20 wt.-%, preferably from 0.1 to 15 wt.-%, and more preferred from 0.5 to 10 wt.-%, based on the total weight of the composition.

Optionally, other conventional active substances, adjuvants or additives, customarily used for disinfectants or antimicrobial preparations may be added in order to obtain a ready-for-use preparation or formulation such as antimicrobial cosmetic or pharmaceutical preparation, such as pharmaceutical drugs, insecticidal compounds, anti-inflammatory agents, vitamins, mineral, skin conditioning agents, fragrances, etc.

The pharmaceutical drugs are preferably selected from the group comprising or consisting of penicillin's such as penicillin, penicillin G, hetacillin potassium, cioxacillin benzathine, ampicillin and amoxicillin trihydrate, aminocoumarins such as novobiocin, cephalosporins such as cephalexin, ceftiofur sodium, ceftiofur hydrochloride, ceftiofur crystalline free acid, macrolides such as tildipirosin, tylosin, tulathromycin, erythromycin, clarithromycin, and azithromycin, quinolones and fluoroquinolones such as enrofloxacin, ciprofloxacin, levofloxacin, and ofloxacin, sulfonamides such as sulfadimethoxine, co-trimoxazole and trimethoprim, retinoids such as retinol, retinal, tretinoin (retinoic acid), isotretinoin, alitretinoin, etretinate, adapalene, bexarotene, and tazarotene, trifarotene, tetracyclines such as tetracycline, oxytetracycline and doxycycline, aminoglycosides such as dihydrostreptomycin sulfate, neomycin, gentamicin and tobramycin, lincosamides such as pirlimycin hydrochloride, lincomycin, clindamycin, and pirlimycin, and amphenicols such as florfenicol, antiviral drugs such as peramivir, zanamivir, oseltamivir phosphate, baloxavir marboxil, acyclovir, valacyclovir, valganciclovir, brivudin, cidofovir, famciclovir, fomivirsen, foscarnet, ganciclovir, penciclovir, amantadine, rimantadine, ribavirin, telbivudine, adefovir, entecavir, emtricitabine, lamivudine, tenofovir, boceprevir, telaprevir, interferons, antimycotic drug is selected from polyenes such as amphotericin b, candicidin, filipin, hamycin, natamycin, nystatin, and rimocidin; azoles such as imidazole, triazole, thiazole, bifonazole, butoconazole, clotrimazole, econazole, fenticonazole, isoconazole, ketoconazole, luliconazole, miconazole, omoconazole, oxiconazole, sertaconazole, sulconazole, tioconazole, albaconazole, efinaconazole, epoxiconazole, fluconazole, isavuconazole, itraconazole, posaconazole, propiconazole, ravuconazole, terconazole, voriconazole, and abafungin; allylamines such as amorolfin, butenafine, naftifine, and terbinafine; echinocandins such as anidulafungin, caspofungin and micafungin; aurones, benzoic acid, ciclopirox olamine, flucytosine or 5-fluorocytosine, griseofulvin, haloprogin, tolnaftate, undecylenic acid, triacetin, crystal violet, castellani's paint, orotomide (f901318), miltefosine, potassium iodide, coal tar, copper(ii) sulfate, selenium disulfide, sodium thiosulfate, piroctone olamine, iodoquinol, clioquinol, acrisorcin, zinc pyrithione, and sulfur, antiprotozoals such as melarsoprol, eflomithine, metronidazole, tinidazole, miltefosine, antihelminthics such as mebendazole, pyrantel pamoate, thiabendazole, diethylcarbamazine, ivermectin, aticestodes such as niclosamide, praziquantel, albendazole, antitrematodes such as praziquantel, antiamoebics such as rifampin and amphotericin B, and broad-spectrum drugs such as nitazoxanide, or a combination thereof.

The vitamins are selected from the group comprising or consisting of retinol (vitamin A), thiamine (vitamin BI), riboflavin (vitamin B2), niacin (vitamin B3), pantothenic acid (vitamin B5), pyridoxine (vitamin B6), biotin (vitamin B7), folic acid (vitamin B9), ascorbic acid (vitamin C), ergocalciferol (vitamin DI), and tocopherols (vitamin E) or a combination thereof for medical or cosmetic applications.

In a preferred variant, the composition according to the present invention comprises
(a) 0.001 to 2.0 wt.-% of the at least one quaternary compound, preferably 0.01 to 0.5 wt.-% of the at least one quaternary compound;
(b) 0.0001 to 15 wt.-% of the at least one alcohol and/or polyol, preferably 0.0001 to 8 wt.-% of the at least one alcohol and/or polyol;
(c) 0.0001 to 4 wt.-% of the at least one inorganic and/or organic acid, preferably 0.0001 to 2 wt.-% of the at least one inorganic and/or organic acid; and
(d) 0.0001 to 5 wt.-% of the at least one essential oil, preferably 0.0001 to 0.3 wt.-% of the at least one essential oil.

In a more preferred variant, the composition according to the present invention comprises
(a) 0.001 to 2 wt.-% of the at least one quaternary compound, preferably 0.01 to 0.5 wt.-% of the at least one quaternary compound, wherein the quaternary compound is selected from the group consisting of of N1-hexadecyl-N1,N1,N2,N2-tetramethyl-ethane-1-diaminium chloride, N1-octadecyl-N1,N1,N2,N2-tetramethyl-ethane-1-diaminiumchloride, N1-hexadecyl-N1,N1,N2,N2-tetramethyl-N2-(3-(trihydroxysilyl-)propyl-)ethane-1,2-diaminium chloride and N1-octacecyl-N1,N1,N2,N2-tetramethyl-N2-(3-(trihydroxysilyl-)propyl-)ethane-1,2-diaminium chloride;
(b) 0.0001 to 15 wt.-% of the at least one alcohol and/or polyol, preferably 0.0001 to 8 wt.-% of the at least one alcohol and/or polyol, wherein the alcohol or polyol is selected from the group consisting of ethanol, n-propanol, 2-propanol, n-butanol, 2-butanol, 3′-butanol, tert.-butanol, 2-methyl-1-butanol, 3-methyl-1-butanol, 3-methyl-2-butanol, 3-methoxy-3-methyl-1-butanol, glycerol, propylene glycol, butylene glycol, pentylene glycol, hexylene glycol, pentaerythritol, trimethylolpropane, 1,2-propanediol, butanediol, pentanediol, hexanediol, diethylene glycol, dipropylene glycol, 2-methyl propanediol, 1,4-butanediol, and any mixture thereof;
(c) 0.0001 to 4 wt.-% of the at least one inorganic and/or organic acid, preferably 0.0001 to 2 wt.-% of the at least one inorganic and/or organic acid, wherein the inorganic acid or organic acid is selected from the group consisting of salicylic acid, sorbic acid, lactic acid, benzoic acid, anisic acid, citric acid, acetylsalicylic acid, ferulic acid, mandelic acid, levulinic acid, hydroxamic acid, caprylic acid, caproic acid, capric acid, citronellic acid, shikimic acid, carbamic acid, cinnamic acid, 2,5-dihydroxybenzoic acid, 3,4-dihydroxybenzoic acid, and any mixture thereof, and salts and derivatives of the afore-mentioned acids as defined above; and
(d) 0.0001 to 5 wt.-% of the at least one essential oil, preferably 0.0001 to 0.3 wt.-% of the at least one essential oil, wherein the essential oil is selected from the group consisting of Rosmarinus officinalis oil (CAS-No: 8000-25-7), Origanum oil (CAS-No.: 8007-11-2), Salvia officinalis oil (CAS-No: 8022-56-8), Bakuchiol suaveolens oil, Cinnamomum cassia bark oil, Pistacia lenticus gum oil (CAS-No: 61789-92-2), Pistacia lenticus leaf oil (CAS-No: 90082-82-9), Artemisia absinthium oil, Melaleuca alternifolia oil (CAS-No.: 68647-73-4), Mentha piperita (peppermint) oil, Mentha spicata oil, Mentha suaveolens oil, Pistacia lenticus gum, Salvia triloba oil, Thymus vulgaris oil (CAS-No.: 84929-51-1), Ocimum basilicum oil (CAS-Nr. 84775-71-3), and any mixture thereof.

In a still more preferred variant, the composition according to the present invention comprises
(a) 0.001 to 2 wt.-% of a mixture of N1-hexadecyl-N1,N1,N2,N2-tetramethyl-ethane-1-diaminium chloride, N1-octadecyl-N1,N1,N2,N2-tetramethyl-ethane-1-diaminiumchloride, N1-hexadecyl-N1,N1,N2,N2-tetramethyl-N2-(3-(trihydroxysilyl-)propyl-)ethane-1,2-diaminium chloride and N1-octacecyl-N1,N1,N2,N2-tetramethyl-N2-(3-(trihydroxysilyl-)propyl-)ethane-1,2-diaminium chloride, preferably 0.01 to 0.5 wt.-%;
(b) 0.0001 to 15 wt.-% pentylene glycol, preferably 0.0001 to 8 wt.-%;
(c) 0.0001 to 4 wt.-% of benzoic acid and/or salicylic acid or salts or derivatives of the afore-mentioned acids as defined above, preferably 0.0001 to 2 wt.-%; and
(d) 0.0001 to 5 wt.-% of Pistacia lenticus gum oil (CAS-No: 61789-92-2) and/or Pistacia lenticus leaf oil (CAS-No: 90082-82-9), preferably 0.0001 to 0.3 wt.-%.

Surprisingly, it revealed that the composition according to the present invention possesses a synergistic antimicrobial efficacy against a broad spectrum of microorganism, i.e., antibacterial, antiviral, antifungal, and antiparasitic efficacy, as it is demonstrated in the following examples.

The synergistic antimicrobial efficacy against a broad spectrum of microorganism is particularly pronounced for a composition formulation as defined before, which comprises a mixture of N1-hexadecyl-N1,N1,N2,N2-tetramethyl-ethane-1-diaminium chloride, N1-octadecyl-N1,N1,N2,N2-tetramethyl-ethane-1-diaminiumchloride, N1-hexadecyl-N1,N1,N2,N2-tetramethyl-N2-(3-(trhydroxysilyl-)propyl-)ethane-1,2-diaminium chloride and N1-octacecyl-N1,N1,N2,N2-tetramethyl-N2-(3-(trihydroxysilyl-)propyl-)ethane-1,2-diaminium chloride; pentylene glycol; benzoic acid and/or salicylic acid; and Pistacia lenticus gum oil (CAS-No: 61789-92-2) and/or Pistacia lenticus leaf oil (CAS-No: 90082-82-9).

Thus, the composition according to the present invention, having a broad-spectrum antimicrobial effect, renders all pathogenic germs harmless with just one formulation.

In particular, the composition according to the present invention has an improved broad spectrum antimicrobial effect despite a lower content of quaternary organic ammonium compounds compared to the hand sanitizer according to the prior art, comprising a mixture of N1-hexadecyl-N1,N1,N2,N2-tetramethyl-N2-(3-(trihyroxysilyl-)propyl-)ethane-1,2-diaminium dichloride, N1,N1,N2,N2-Tetramethyl-N1-octadecyl-N2-(3-(trihydroxysilyl-)propyl-)ethane-1,2-diamonium dichloride and octadecyldimethyl-(3-trihydroxysilylpropyl-)ammonium chloride in combination with high concentrations of additional differently structured quaternary organic ammonium compounds, namely benzalkonium chloride (0.13 wt.-%), lauramine oxide (0.13 wt.-%) and didecyldimethylammoniumchloride (0.5 wt.-%), i.e. with a total content of these quaternary organic ammonium compounds of 0.76 wt.-%. However, benzalkonium chloride, lauramine oxide and didecyldimethylammoniumchloride are highly toxic and irritant to human skin, eyes, to animals and to the environment, in particular in these concentrations.

The composition according to the presented invention kills a wide range of microorganism immediately after application, i.e., the composition develops its full effect with the shortest possible exposure time and is fast effective at the time of use.

Furthermore, it turned out that the composition according to the present invention exhibits an improved long-lasting antimicrobial activity, i.e., an antimicrobial effect for at least 48 h. Hence, with the use of the composition according to the present invention protection of surfaces can be continued and re-contamination with microorganism can be prevented for a prolonged period.

In addition, the composition according to the present invention has a low toxicity for humans, but also animals and the environment. The low toxicity is attributed to the eco-friendly ingredients of the composition on the one hand and to the lower total concentration of the quaternary compounds in the composition.

Furthermore, the composition can be prepared at a large scale and by simply mixing the ingredients.

Due to the described advantageously properties, the composition according to the first aspect of the present invention can be used for the preparation of a disinfectant or an antimicrobial preparation, preferably for a cosmetic or pharmaceutical preparation.

Hence, the present invention relates in a second aspect to a disinfectant or an antimicrobial preparation comprising the composition according to the present invention. In a preferred variant, the antimicrobial preparation is a cosmetic or pharmaceutical preparation.

In a preferred variant, the preparation according to the present invention is a preparation for cosmetic and/or non-therapeutic use for personal care, skin protection, skin care, scalp protection, scalp care or nail care. Examples of personal care are preferably anti-ageing preparations, skin care emulsions, body oils, body lotions, cleansing lotions, face, or body balms, after shave balms, after sun balms, deo emulsions, cationic emulsions, body gels, treatment creams, skin protection ointments, moisturizing gels, face and/or body moisturizers, light protective preparations (sunscreens), micellar water, hair spray, colour protection hair care products, skin lightening product, anti-dark spot preparations, etc.

Due to their outstanding antimicrobial efficacy as described above and demonstrated by the following examples, the composition according to the first aspect and the antimicrobial preparations according to the second aspect of the present invention is suitable for sanitizing or disinfecting, in particular sanitizing or disinfecting surfaces, skin, or objects.

When a product claims to sanitize a surface, it is promising to reduce the level of germs that could be harmful to your health to meet to public health standards or requirements. Sanitizing reduces, not kills, the number and growth of bacteria, viruses, and fungi. Sanitizing is particularly important in food preparation areas where germs and fungi can cause foodborne illnesses.

By contrast, the act of disinfecting kills microscopic organisms (bacteria, viruses, fungi) on surfaces. Disinfection is usually achieved by using EPA-approved chemicals that kill the organisms and prevent them from spreading.

Thus, in a further aspect the present invention relates to the use of the composition according to the invention or the disinfectant or antimicrobial preparation for suppressing or inhibiting microorganism growth, i.e., to kill and prevent bacterial, virus, fungal and parasitic deposition, formation, spreading and survival on surfaces, on skin or on objects. The suppression or inhibition of the growth of microorganisms is a reduction of the microorganisms' growth rate and/or stagnation or reduction of the number of living microorganisms.

Preferably, the disinfectant is used for disinfecting or sanitizing surfaces selected from the group consisting of plastics, metal, steel, wood, ceramics, tissue, glass, granite, stone, marble, cloths, fabrics or medical or surgical equipment. These surfaces can be found in private and public spaces such as retails, hospitals, hotels, amusement parks, educational facilities, such as schools, universities, factories, boats, airports, planes interiors and exteriors, metro and train interiors and exteriors, buses interiors and exteriors, cars' interiors and exteriors, toilets, bathrooms, etc., i.e., surfaces with can be contaminated with harmful microorganism.

In a preferred variant, the composition according to the present invention is used for disinfecting or sanitizing objects such as medical or surgical devices, endo-objects who stay in the human body for a longer period, such as catheters, redon drains.

In a preferred variant, the composition according to the present invention is used in the prevention or treatment of a disorder in a mammal, such as a human, by coating skin, hair and/or nails.

Alternatively, the preparation according to the present invention is a preparation for use in prevention or treatment of disorder in a mammal, such as a human, preferably a skin disorder in a mammal, such as a human.

For use in the prevention and treatment of a disorder in a mammal, such as a human, the preparation is applied to animated surfaces such as human or animal tissues, such as skin, mucosa, nails, hair, bones, intraperitoneal tissues, intracranial tissues, intrapelvic tissues, intramediastinal tissues, intrapericardial tissues, during any type of surgical procedure, surgical wounds, skin wounds, mucosal wounds, skin diseases (eczema, acne vulgaris, acne rosacea), topic infectious diseases or burn injuries.

The use of the antimicrobial, i.e., antibacterial, antiviral, antifungal and antiparasitic, composition or disinfectant or antimicrobial preparation according to the present invention as defined herein is for killing a broad spectrum of microbials, namely gram-positive bacterial, gram-negative bacteria, viruses, non-enveloped and enveloped viruses, moulds, mildews, yeast, fungi, and parasites.

A use is further preferred, wherein the microorganisms are selected from the group consisting of bacteria selected from the group consisting of St. aureus, E. coli, Pseudomonas aeruginosa, Methicillin-resistant Staphylococcus aureus (MRSA), St. epidermidis, Enterobacter cloacae, Enterococcus faecalis, Corynebacterium, Clostridium, Listeria, Bacillus, S. saprophyticus, S. pyogenes, S. agalactiae, Saccharomyces cerevisiae, Aspergillus brasiliensis, S. pneumoniae, S. viridans, Salmonella, Shigella, Moraxella, Helicobacter, Stenotrophomonas, Bdellovibrio, Legionella, Neisseria, Gonorrhoeae, Neisseria meningitidis, Moraxella catarrhalis, Haemophilus influenzae, Proteus mirabilis, Enterobacter cloacae, Serratia marcescens, Helicobacter pylori, Salmonella enteritidis, Salmonella typhi, Acinetobacter baumannii, and Mycobacterium tuberculosis;
viruses, non-enveloped and enveloped viruses, selected from the group consisting of coronavirus such as SARS-Cov-1 and SARS-Cov-2, SARS, Middle East respiratory syndrome (MERS), influenza A, B and C virus, hantavirus, Anders hantavirus, human parainfluenza viruses, respiratory syncytial virus, marburg virus, ebola virus, rabies, human immunodeficiency virus (HIV), smallpox, dengue, rotavirus A, B and C, human papillomaviruses (HPVs), Epstein-Barr virus (EBV), hepatitis A, B and C virus, human herpes virus (HHV), human T-lymphotropic virus-1 (HTLV-1), merkel cell polyomavirus (MCV), Equine Arteritis virus, simian virus 40 (SV40), adeno-associated virus, aichi virus, measles virus and yellow fever virus, mould, mildew; and fungi or parasites selected from the group comprising protozoa, nematodes, necrotrophs, biothrophs, and worms such as Chrysomyxa ledi, Phragmidium, Taphrina Confusa, Epichloe typhina, Armillaria, striga, Albugo Candida, Plasmodiophora brassicae, powdery mildews, downy mildews, Pythium species, Sclerotinia rots (S. sclerotiorum, S. minor, Sclerotium rolfsii and S. cepivorum), Fusarium wilts and rots (various Fusarium species including F. solani and F. oxysporum), Botrytis rots (Botrytis cinerea), anthracnose (Colletotrichum spp. and Microdochium panattonianum), Rhizoctonia rots (Rhizoctonia solani), damping-off (Pythium, Rhizoctonia, Phytophthora, Fusarium or Aphanomyces), cavity spot (Pythium sulcatum), tuber diseases, rusts, such as Puccinia sorghi, Uromyces appendiculatus, Puccinia allii, black root rot, Alternaria solani, Aphanomyces euteiches pv. Phaseoli, Aschochyta collar rot, Didymella bryoniae, Alternaria cucumerina and A. altemata, Leptosphaeria maculans, Mycosphaerella brassicicola, Septoria apiicola, Cercospora beticola, Septoria petroelini, Septoria lactucae, Stemphylium vesicarium, Alternaria dauci, Candida, ringworm, Blastomyces, Coccidioides, Histoplasma, Cryptococcus gattii, Neoformans, Mucormycetes, Cryptococcus Paracoccidioides, Aspergillus, Taloromyces, Pneumocystis jirovecii, Candida auris, Candida albicans, Sporothrix, Trichophyton rubrum, dermatophytes that cause tinea cruris, tinea corporis, tinea pedis, tinea Versicolor, Tinea unguium, Tinea faciei, Tinea manuum, and Tinea capitis in mammals such as Trichophyton rubrum, Trichophyton tonsurans, Trichophyton mentagrophytes, Trichophyton verrucosum, Trichophyton schoenlenii, Trichophyton equinum, Trichophyton kanei, Trichophyton raubitschekii, Trichophyton violaceum, Microsporum canis, Microsporum audouinii, Microsporum gypseum, Microsporum equinum, Microsporum nanum, Microsporum versicolor, and Epidermophyton floccosum, and archaea, algae and germs, or any combinations thereof.

The composition according to the first aspect of the present invention and the disinfectant or antimicrobial preparation showed excellent efficacy against these microorganisms.

Surprisingly, the composition according to the present invention comprising a mixture of N1-hexadecyl-N1,N1,N2,N2-tetramethyl-ethane-1-diaminium chloride, N1-octadecyl-N1,N1,N2,N2-tetramethyl-ethane-1-diaminiumchloride, N1-hexadecyl-N1,N1,N2,N2-tetramethyl-N2-(3-(trihydroxysilyl-)propyl-)ethane-1,2-diaminium chloride and N1-octacecyl-N1,N1,N2,N2-tetramethyl-N2-(3-(trihydroxysilyl-)propyl-)ethane-1,2-diaminium chloride; pentylene glycol; benzoic acid and/or salicylic acid; and Pistacia lenticus gum oil (CAS-No: 61789-92-2) and/or Pistacia lenticus leaf oil (CAS-No: 90082-82-9) has been found to have a synergistically enhanced antimicrobial effect.

In a preferred variant, the antimicrobial, i.e., antibacterial, antiviral, antifungal and antiparasitic, composition or disinfectant or antimicrobial preparation according to the present invention is used for killing Staphylococcus aureus, Enterococcus hirae, Pseudomonas aeruginosa, Escherichia coli, Candida albicans, Aspergillus brasiliensis and/or Saccharomyces cerevisiae.

In a preferred variant, the antimicrobial, i.e., antibacterial, antiviral, antifungal and antiparasitic, composition or disinfectant or antimicrobial preparation according to the present invention is used for killing SARS-Cov-1 and 2.

The composition according to the first aspect of the present invention and the disinfectant or antimicrobial preparation according to the second aspect of the present invention showed excellent antimicrobial efficacy against these microorganisms, as it is demonstrated by the following examples.

In particular, the composition according to the present invention comprising a mixture of N1-hexadecyl-N1,N1,N2,N2-tetramethyl-ethane-1-diaminium chloride, N1-octadecyl-N1,N1,N2,N2-tetramethyl-ethane-1-diaminiumchloride, N1-hexadecyl-N1,N1,N2,N2-tetramethyl-N2-(3-(trihydroxysilyl-)propyl-)ethane-1,2-diaminium chloride and N1-octacecyl-N1,N1,N2,N2-tetramethyl-N2-(3-(trihydroxysilyl-)propyl-)ethane-1,2-diaminium chloride; pentylene glycol; benzoic acid and/or salicylic acid; and Pistacia lenticus gum oil (CAS-No: 61789-92-2) and/or Pistacia lenticus leaf oil (CAS-No: 90082-82-9) has been found to have a synergistically pronounced antimicrobial effect.

In a further aspect the present invention also relates to a method for disinfecting or sanitizing a surface, skin or an object comprising the steps of
applying an effective amount of the composition as defined herein or the disinfectant or antimicrobial preparation as defined herein onto a surface, skin, or object; and
contacting the surface, skin or the object with the composition, disinfectant, or antimicrobial preparation for a contact time of at least 15 seconds.

The composition as defined herein or the disinfectant or antimicrobial preparation as defined herein can easily applied to a surface, skin or to an object through various application methods, such as spraying, as pressurized aerosol, by fogging, rolling, brushing, mopping, wiping, dipping, injecting or any combination thereof.

After application of the composition according to the first aspect of the present invention or the disinfectant or antimicrobial preparation according to the second aspect of the present invention, the solvent or the mixture of solvents evaporate quickly and, thus, promote the formation of a film on a surface, skin, or an object. The thus formed antimicrobial coating prevents the surfaces from re-contamination with microbes for a prolonged period, thus, making frequent disinfection or sanitizing superfluous.

In a preferred variant, the contact time is 30 minutes to efficiently kill the microorganism or prevent microorganism growth.

In a final aspect, the present invention relates to a method for preparing a quaternary compound according to general formula (I) as defined herein.

Said method corresponding to a first synthesis step, comprises the steps of
mixing Y1,Y1,Y2,Y2-Tetra-(alkyl-laryl-)alkylendipnictogenin with a n-halogen alkane,
heating the reaction mixture to a temperature of at least 60° C.;
letting the mixture react for at least 4 days at a temperature of at least 60° C. in order to obtain the mono-cation; and
separating the Y1-(alkyl-laryl-)Y1,Y1,Y2,Y2-tetra-(alkyl-/aryl-)alkane-1-dipnictogeninium halogenide;
according to the following reaction schemata:

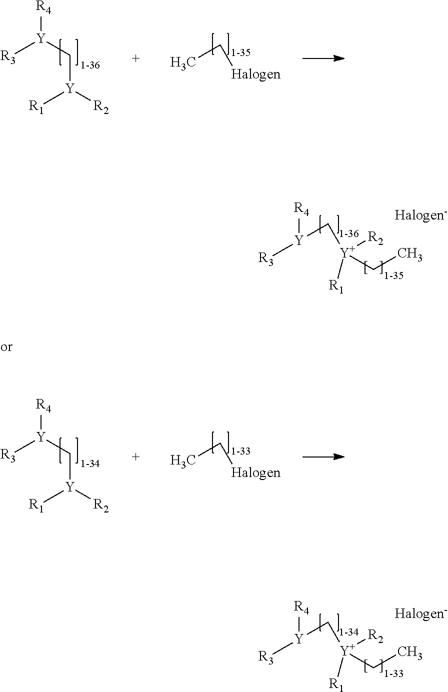

wherein
   Y represents phosphor or nitrogen;
   R₁ and R₂ are independently from each other selected from the group consisting of H, C1 to C36 n-alkyl, C1 to C36 i-alkyl, C1 to C36 sec-alkyl, C1 to C36 t-alkyl, aryl or C1 to C36 t-alkyl-silyl;
   R₃ and R₄ are independently from each other selected from the group consisting of H, C1 to C36 n-alkyl, C1 to C36 i-alkyl, C1 to C36 sec-alkyl, C1 to C36 t-alkyl, aryl and C1 to C36 t-alkyl-silyl; and
   halogen is selected from the group consisting of F, Cl, Br and I.

In an alternative variant, the present invention relates to a method for preparing a quaternary compound according to the general formula (II) as defined herein.

To obtain the quaternary compound according to the general formula (II) a further, i.e., second synthesis step is carried out following the first synthesis step as described before. The method according to the second synthesis steps comprising the steps of
mixing Y1-(alkyl-/aryl-)Y1,Y1,Y2,Y2-tetra-(alkyl-laryl-)alkane-1-dipnictogeninium halogenide with a n-halogenoalkyl-)trialkoxysilan;
heating the reaction mixture to a temperature of at least 60° C.;
letting the mixture react for at least 4 days at a temperature of at least 60° C. in order to obtain the di-cation; and
separating the Y1-(alkyl-/aryl-)Y1,Y1,Y2,Y2-tetra-(alkyl-/aryl-)-Y2-(3-[tri(alkyloxy-/hydroxy-)silyl-]alkyl)alkane-1,2-dipnictogeninium halogenide;
according to the following reaction schemata:

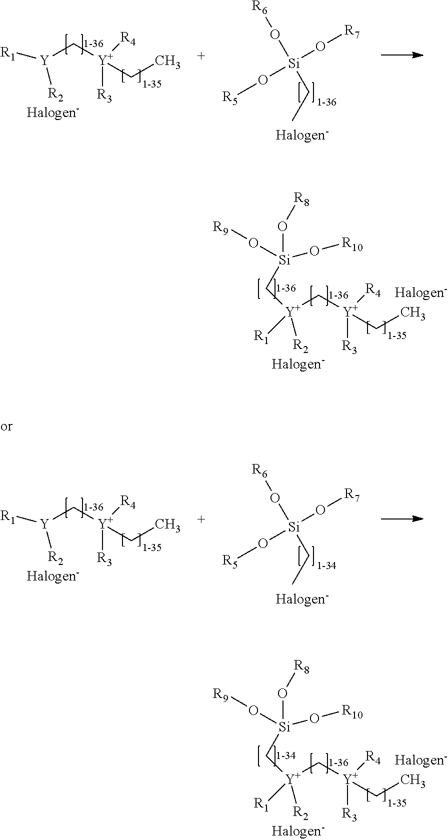

wherein
   Y represents phosphor or nitrogen;
   R₁ and R₂ are independently from each other selected from the group consisting of H, C1 to C36 n-alkyl, C1 to C36 i-alkyl, C1 to C36 sec-alkyl, C1 to C36 t-alkyl, aryl or C1 to C36 t-alkyl-silyl;
   R₃ and R₄ are independently from each other selected from the group consisting of H, C1 to C36 n-alkyl, C1 to C36 i-alkyl, C1 to C36 sec-alkyl, C1 to C36 t-alkyl, aryl and C1 to C36 t-alkyl-silyl;
   R₅, R₆ and R₇ are independently from each other selected from the group consisting of H, C1 to C36 n-alkyl, C1 to C36 i-alkyl, C1 to C36 sec-alkyl, C1 to C36 t-alkyl and aryl; and
   halogen is selected from the group consisting of F, Cl, Br and I.

The compounds according to the general formula (II) obtained by the second synthesis steps react during the purification process with humidity on their silylation-functions by elimination of ethanol. Effectively, there will result a mixture of different compounds with different hydrolysis state.

Basically, one allows an N,N,N′,N′-tetraalkylated alkylenediamine to react with alkyl halide in the first synthesis step, thereby synthesizing the mono-cation with monohalide counterion.

In the second synthesis step, about 50% of the mono-cation monohalide synthesized of the first synthesis step is then further reacted with n-haloalkyl trialkoxysilane to synthesize the di-cation with dihalide counterion. However, the mono-cation monohalide which was not converted in the second synthesis step need not to be isolated from the second synthesis step products by purification steps after completion of the second synthesis step reaction, because the mono-cation portion not converted in the second synthesis step also has superior microbiocidal properties and very low toxicity.

The preparation of the compounds according to general formula (I) and formula (II) is as follows:

First synthesis step for the synthesis of the mono-cation of the compound of the general formula (I):

### Educt Chemicals:

8.54 g (11.09 ml) TMEDA {(73.51 mmol, 2.20 equiv.) CAS-no.:110-18-9; density=0.77 g/ml} must be in colorless quality, otherwise TMEDA must be precleaned by distillation;
4.82 g (=5.67 ml) of 1-chlor-Octadecane {(16.675 mmol, 0.5 equiv.) CAS-no.:3386-33-2; density=0.849 g/ml};
4.35 g (=5.03 ml) of 1-chlor-Hexadecane {(16.675 mmol, 0.5 equiv.) CAS-no.: 4860-03-1; density=0.865 g/ml};
Toluene pre-dried over 3 Å molecular sieve {CAS-no.: 108-88-3; density=0.87 g/ml; bp=110° C.};
tert.-butyl methyl ether (MTBE) pre-dried over 3 Å molecular sieve {CAS-no.: 1634-04-4; density=0.74 g/ml; bp=55° C.};
optional recrystallizing solvent acetonitrile (MeCN) pre-dried over 3 Å molsieve {CAS-no.: 75-05-8; 41.05 g/mol; density=0.78 g/ml; bp=82° C.}.

Place, under medium strong argon-flow, the TMEDA and the two alkyl halogenides together with 32.2 g Toluene in the flask. Then heat-up the reaction mixture to 80° C. flask temperature. Then let the reaction mixture react during 5 days at 80° C. (flask temperature) under low to medium strong argon-flow. By then the first step reaction should be finished. Cool down the reaction mixture to 50° C., then change the double-deck format serpentine condenser (under strong argon flow) against a Liebig cooler condenser with distillation/connectors with additional condensate flask. Then distill off as much solvent as possible (until getting a nearly instirrable slurry) under reduced pressure (for remaining toluene/water and excess of TMEDA as much as possible). Then cool down to 50° C. and additionally add approx. 25 ml of dry toluene trough the dropping funnel as liquid compensation and then distill away as much as possible under reduced pressure (until getting a nearly instirrable slurry). Repeat this toluene adding and removing (in 3 additional portions A 25 ml each) three times (now all of the excessed TMEDA should be removed by distillation over Liebig condenser, and you should have a nearly instirrable slurry. Let it cool down to approx. 40° C. and equalize the pressure by argon compensation. Refill the dropping funnel under medium strong Argon flow with approx. 75 ml of total dry MTBE. Clean-up the slurry 3 times. Repeating cleaning steps as following described: add 25 ml of fully dry MTBE, then heat it up until it begins to boil, then cool it down (0° C.), then filter as much liquid as possible through filter candle, or better by centrifugation (under very strong argon flow). Now a suitable clean, total humidity-free, nearly white first step product {=mono cation ═N1-C16/C18-alkyl-N1,N1,N2,N2-tetramethyl-ethane-1-diaminium chloride should be obtained as slurry in residual MTBE with traces of toluene and traces of Beta-Hoffmann-Elimination-by product=TMEDA-hydrochloride, which will be removable by recrystallization-purification in Acetonitrile after the second step reaction. This mono cation-slurry is the starting material for the second step reaction. The slurry of mono cation is very, very hygroscopic and must be strictly handled under absolute dry conditions, otherwise a non-suitable, rubber product will be obtained!!! For the determination of the mono cation-content in the obtained slurry, which is essential for the second step reaction, a little part of slurry, must be gravimetric dried at room temperature conditions in high-vacuum ≤0.01 mbar. In the first-step reaction a yield of in minimum 56%, calculated on placed chlor-alkane-mixture, start material should be obtained.

Second synthesis step for the synthesis of the di-cation of the compound of the general formula (II) together with unreacted compounds of the first synthesis step:

### Educt Chemicals:

Mono-cation slurry obtained from the first synthesis step calculated as pure mono-cation, according gravimetric- and HPLC-analysed mono-cation content determination; 3 equivalents calculated on the basis of the pure mono-cation of 3-chlorpropyl-triethoxysilan {CAS-no.:5089-70-3; 240.80 g/mol; density=1.00 g/ml; bp=221° C.};
70 equivalents calculated on the basis of the pure mono-cation of acetonitrile (MeCN) pre-dried over 3 Å mol sieve {CAS-no.:75-05-8; 41.05 g/mol; density=0.78 g/ml; bp=82° C.};
40 equivalents calculated on the basis of the pure mono-cation of n-heptane pre-dried over 3 Å mol sieve {CAS-no.: 142-82-5; 100.21 g/mol; density=0.68 g/ml; bp=98° C.};
6 equivalents calculated on the basis of the pure mono-cation as co-vapor ethanol pre-dried over 3 Å mol sieve {CAS-no.: 67-17-5; 46.07 g/mol; density=0.7893 g/ml; bp=78.3° C.};
2.52 equivalents calculated based on the pure mono-cation as adjust ethanol pre-dried over 3 Å mol sieve {CAS-no.: 67-17-5; 46.07 g/mol; density=0.7893 g/ml; bp=78.3° C.}.

Removal of any possible humidity contamination from the products (mono-cations) resulting from the first synthesis step.

Place the mono-cation slurry under strict dry conditions and under strong argon-flow in a pressure stable vessel. If there is a trace of moisture in the mono quaternary salt, you may add ˜1 ml of MeOSiMe3 or EtOSiMe3 per gram of mono-cation as followed described. Then spill any gauge vent needle together with a mounted syringe (for later adding of MeOSiMe3 or EtOSiMe3) through the rubber-septa (on the vessel) and fill the syringe 3-times with argon-atmosphere of the vessel content (press out and refill 3 to 5-times, to get out all of possible syringe moisture), then stitch the needle of the full syringe (fully filled with argon) through the septa of the (MeOSiMe3 or EtOSiMe3-bottle as much, that the needle will be dipped in liquid [=MeOSiMe3 or EtOSiMe3]. Then empty the syringe completely (=press out the argon) and fill it with liquid (=MeOSiMe3 or EtOSiMe3). Then stitch the syringe needle very fast through the gum septa of the vessel and empty the hole syringe content in the vessel. Then gently heat (˜40° C. with stirring under argon for 15 minutes, then distill off the excess TMS ether, TMS-O-TMS and MeOH/EtOH under reduced pressure until constant weight. Additionally place the MeCN under strong argon-flow to the optionally pre dried mono-cation in the pressure vessel and spill afterwards during 15 minutes under medium strong argon flow. Then add the 3-chlorpropyl-triethoxysilan as followed described to the mono-cation solution (in MeCN) under still medium strong Argon flow: Spill an clean gauge vent needle together with a mounted clean syringe (for later adding of 3-chlorpropyl-triethoxysilan) through the rubber septa (on the vessel) and fill the syringe 3-times with argon-atmosphere of the vessel content (press out and refill 3 to 5-times, to get out all of possible syringe moisture), then stitch the needle of the fully syringe (fully filled with argon) through the septa of the 3-chlorpropyl-triethoxysilan-bottle as much, that the needle will be dipped in liquid (3-chlorpropyl-triethoxysilan). Then press out the syringe totally (=press out the argon) and fill them with liquid (3-chlorpropyl-triethoxysilan). Then very fasten stitch the syringe needle through the gum septa of the vessel and press out the hole syringe content in the vessel. May be this whole process of spilling, filling and emptying of the syringe has to be repeated several times, until the whole amount of adding (3-chloropropyl)-triethoxysilane could be placed into the vessel. Then heat up the vessel to 115° C. vessel temperature and let them react for 5 days at 115° C. Then cool down the reaction mixture to 75° C. under Argon compensation. Purification of the crude and removal of excess silane and Beta-Hoffmann-Elimination by-product (octadecene) is accomplished as follows. Add 10 Equivalent of dry Heptane under strong Argon flow to the reaction mixture. Then the reaction mixture (incl. heptane) was agitated for several minutes with gentle heating (up to 75° C. vessel temperature). Then stop stirring and let stand until and a clear phase-separation could be observed, and any traces of precipitated Di-cation had settled (lower solvent phase=MeCN-phase contains unreacted mono-cation and the Di-cation product and traces of precipitated Di-cation, together with Beta-Hoffmann-elimination by-product (=TMEDA-hydrochloride); upper solvent phase=heptane and extracted silane excess part. Then separate the hot lower MeCN-phase by ˜75° C. through the bottom ventil on the vessel (by continuously argon-compensation) and collect them temporary in totally humidity-free, argon-spilled, pre-heated other vessel (80° C. jacket-temperature). Then waste the residual upper heptane-phase. This heptane-extraction and separation process (at 75° C.) must be repeated 3 additional times identically with 10 equivalents of heptane per each extraction process (in total to extract with 40 equivalents of heptane in comparison to pure calculate mono-cation). After this 4 times repeated extraction purification, the 3-chloropropyl triethoxysilane excess should be minimized to a very low level, so that unreacted mono-cation and the di-cation (=second synthesis step product) can be precipitated out of MeCN in two crystallization steps as followed. cool down the MeCN-phase to 45° C. under strict humidity-free conditions by argon compensation and let crystallize containing unreacted mono-cation and di-cation at 45° C. Please notice that cooling to 45° C. must happened slowly, because at temperatures lower than 45° C. unreacted mono-cation and di-cation will precipitate out extremely quickly to an undesirable mono-block. Centrifuge or filter the obtained crystallite under strict humidity-free conditions (otherwise a non-suitable, gummi product will be obtained), and wash the filter cake with dry cold (0° C.) MeCN.

Then concentrate the collected filtrate (incl. wash MeCN) under reduced pressure to 50% and cool down the concentrated MeCN-phase to 0° C. under strict humidity-free conditions by argon compensation and let crystallize containing unreacted mono-cation and di-cation at 0° C. Centrifuge or filter the obtained crystallite under strict humidity-free conditions (otherwise a non-suitable, gummi product will be obtained), and wash the filter cake with dry cold (0° C.) MeCN. This filtrate concentration and crystallization step can optionally be repeated for getting higher yield of unreacted mono-cation and di-cation, as long the removing TMEDA-hydrochloride (Beta-Hoffmann-elimination by-product) still is enough soluble in the concentrated, residual MeCN-content at cold (0° C.) conditions!!! For total removing of disliked MeCN residuals in finish product mixture, the collected (MeCN-clammy) filter cake must be solubilized in 2 equivalents (compares to mono-cation start material amount) of absolutely dried (mol sieve-dried) ethanol by afterwards co-evaporation (under reduced pressure and strict humidity-free conditions). This solubilization with 2 equivalent ethanol and following co-evaporation, must be repeated two additional times. With this solubilization with in total 6 equivalents of dry ethanol and following co-evaporation steps, the final MeCN content should be removable to lower then 0.3%. Then the final concentration of storage- and delivery-stable compound-solution must be adjusted by final addition of 2.52 mol equivalent of dry ethanol, calculated on the basis of pure mono-cation start material.

The present invention shall now be described in detail with reference to the following examples, which are merely illustrative of the present invention, such that the content of the present invention is not limited by or to the following examples.

### EXAMPLES

### Example 1: Quantitative Suspension Test for the Evaluation of Bactericidal Activity According to DIN EN 1276 and DIN EN 1650 of BueGEM Hand Sanitizer According to the Present Invention

The tested hand sanitizer had the following composition:

TABLE 1

| Ingredient | Amount |
|---|---|
| (INCI) | (wt.-%) |
| AQUA (WATER) | 85.02116752 |
| PENTYLENE GLYCOL | 7.5 |
| COCAMIDOPROPYL BETAINE | 3.3 |
| DECYL GLUCOSIDE | 1.53 |
| BENZOIC ACID | 0.43 |
| ASCORBIC ACID | 0.02 |
| UREA | 0.2 |
| ROSMARINUS OFFICINALIS (ROSEMARY) OIL | 0.09395 |
| SIMMONDSIA CHINENSIS (JOJOBA) SEED OIL | 0.01 |
| TOCOPHEROL | 0.0007 |
| GLYCINE SOJA OIL UNSAPONIFIABLES | 0.0002 |
| HELIANTHUS ANNUUS (SUNFLOWER) SEED OIL | 0.0001 |
| MENTHA PIPERITA (PEPPERMINT) OIL | 0.04955 |
| CITRIC ACID | 0.931622482 |
| SODIUM CHLORIDE | 0.55 |
| PISTACIA LENTICUS LEAF OIL | 0.1 |
| N1-OCTADECYL-N1,N1,N2,N2-TETRAMETHYL- | 0.0324 |
| ETHANE-1-DIAMINIUM CHLORIDE | |
| N1-HEXADECYL-N1,N1,N2,N2-TETRAMETHYL- | 0.0324 |
| ETHANE-1-DIAMINIUM CHLORIDE | |
| SODIUM BENZOATE | 0.055 |
| N1-OCTADECYL-N1,N1,N2,N2-TETRAMETHYL- | 0.0324 |
| N2-(3-(TRIHYDROXYSILYL)PROPYL)ETHANE- | |
| 1,2-DIAMINIUM CHLORIDE | |
| N1-HEXADECYL-N1,N1,N2,N2-TETRAMETHYL- | 0.0324 |
| N2-(3-(TRIHYDROXYSILYL)PROPYL)ETHANE- | |
| 1,2-DIAMINIUM CHLORIDE | |
| MELALEUCA ALTERNIFOLIA (TEA TREE) LEAF OIL | 0.01896 |
| ALCOHOL | 0.0504 |
| LIMONENE | 0.00655 |
| LINALOOL | 0.0022 |
| Total: | 100.00 |

The antimicrobial efficacy was determined in accordance with DIN EN 1276 (tested microorganisms: Staphylococcus aureus, Enterococcus hirae, Pseudomonas aeruginosa and Escherichia coli) for testing the bactericidal efficacy and in accordance with DIN EN 1650 (tested microorganisms: Candida albicans and Aspergillus brasiliensis) for testing the yeasticidal and fugicidal efficacy.

TABLE 2

| | log-reduction | | | | |
|---|---|---|---|---|---|
| | Duration | | Tested microorganism | | |
| | of action | | S. auereus | C. albicans | A. brasiliensis |
| Hand | 15 | S | >6.5 | >5.5 | <2.6 |
| sanitizer | 30 | s | 5.8 | >5.5 | <2.6 |
| | 60 | s | >6.5 | >5.5 | <2.6 |

Based on DIN EN 1276 and DIN EN 1650 the tested hand sanitizer possessed bactericidal activity when diluted at 80, 50 or 10% (v/v) in water for the referenced strains of Staphylococcus aureus, Candida albicans and Aspergillus brasiliensis.

As can be seen from the above results, the tested hand sanitizer according to the present invention exhibits an antimicrobial broad band effect, including mould, and, thus, fulfils the EU biocide product directive.

In addition, the total concentration of quaternary compounds is less than <0.13%, and, thus, is conform with the cosmetic directive.

Thus, the composition according to the present invention exhibits a broad spectrum antimicrobial effect despite a lower total content of quaternary organic ammonium compounds.

### Comparative Example 1: Quantitative Suspension Test for the Evaluation of Bactericidal Activity According to DIN EN 1276 and DIN EN 1650 of a Surface Disinfectant

The tested surface disinfectant had the following composition:

TABLE 3

| Ingredient | Amount |
|---|---|
| (INCI) | (wt.-%) |
| AQUA (WATER) | 98.507141 |
| N1-OCTADECYL-N1,N1,N2,N2-TETRAMETHYL- | 0.0324 |
| ETHANE-1-DIAMINIUM CHLORIDE | |
| N1-HEXADECYL-N1,N1,N2,N2-TETRAMETHYL- | 0.0324 |
| ETHANE-1-DIAMINIUM CHLORIDE | |
| N1-OCTADECYL-N1,N1,N2,N2-TETRAMETHYL- | 0.0324 |
| N2-(3-(TRIHYDROXYSILYL)PROPYL)ETHANE-1,2- | |
| DIAMINIUM CHLORIDE | |
| N1-HEXADECYL-N1,N1,N2,N2-TETRAMETHYL- | 0.0324 |
| N2-(3-(TRIHYDROXYSILYL)PROPYL)ETHANE-1,2- | |
| DIAMINIUM CHLORIDE | |
| ALCOHOL | 0.0504 |
| BENZALKONIUM CHLORIDE | 0.13 |
| DIDECYLDIMETHYLAMMONIUM CHLORIDE | 1.0 |
| CITRIC ACID | 0.18285905 |
| Total: | 100.00 |

The surface disinfectant was tested in the same manner as described for the hand sanitizer in Example 1. Based on DIN EN 1276 and DIN EN 1650 the tested surface disinfectant possessed bactericidal activity when diluted at 80%, 50% or 10% (v/v) in water for the referenced strains of Staphylococcus aureus, Candida albicans and Aspergillus brasiliensis.

The tested surface disinfectant according to the present invention exhibits an antimicrobial broad band effect, including mould, and, thus, fulfils the EU biocide product directive.

However, the antimicrobial broad band effect can only be obtained with the addition of benzalkonium chloride (0.13 wt.-%) and didecyldimethylammonium chloride (1.0 wt.-%), i.e. an increased total amount of quaternary compounds, which amounts to >1.25 wt.-%. However, benzalkonium chloride and didecyldimethylammonium chloride are toxic and irritant to human skin, eyes, to animals and to the environment. In addition, the total amount of quaternary compounds exceeds the maximum permissible value of 0.13 wt.-%, and, thus, is not conform with the cosmetic directive.

### Comparative Example 2: Quantitative Suspension Test for the Evaluation of Bactericidal Activity According to DIN EN 1276 and DIN EN 1650 of a Surface Disinfectant

The tested surface disinfectant had the following composition:

TABLE 4

| Ingredient | Amount |
|---|---|
| (INCI) | (wt.-%) |
| AQUA (WATER) | 98.377141 |
| N1-OCTADECYL-N1,N1,N2,N2-TETRAMETHYL- | 0.0324 |
| ETHANE-1-DIAMINIUM CHLORIDE | |
| N1-HEXADECYL-N1,N1,N2,N2-TETRAMETHYL- | 0.0324 |
| ETHANE-1-DIAMINIUM CHLORIDE | |
| N1-OCTADECYL-N1,N1,N2,N2-TETRAMETHYL- | 0.0324 |
| N2-(3-(TRIHYDROXYSILYL)PROPYL)ETHANE- | |
| 1,2-DIAMINIUM CHLORIDE | |
| N1-HEXADECYL-N1,N1,N2,N2-TETRAMETHYL- | 0.0324 |
| N2-(3-(TRIHYDROXYSILYL)PROPYL)ETHANE- | |
| 1,2-DIAMINIUM CHLORIDE | |
| ALCOHOL | 0.0504 |
| BENZALKONIUM CHLORIDE | 0.13 |
| LAURAMINE OXIDE | 0.13 |
| DIDECYLDIMETHYLAMMONIUM CHLORIDE | 1.0 |
| CITRIC ACID | 0.18285905 |
| Total: | 100.00 |

The surface disinfectant was tested in the same manner as described for the hand sanitizer in Example 1. Based on DIN EN 1276 and DIN EN 1650 the tested surface disinfectant possessed bactericidal activity when diluted at 80%, 50% or 10% (v/v) in water for the referenced strains of Staphylococcus aureus, Candida albicans and Aspergillus brasiliensis.

The tested surface disinfectant according to the present invention exhibits an antimicrobial broad band effect, including mould, and, thus, fulfils the EU biocide product directive.

However, the antimicrobial broad band effect can only be obtained with the addition of benzalkonium chloride (0.13 wt.-%) and didecyldimethylammonium chloride (1.0 wt.-%), i.e. an increased total amount of quatermary compounds, which amounts to >1.38 wt.-%. However, benzalkonium chloride and didecyldimethylammonium chloride are toxic and irritant to human skin, eyes, to animals and to the environment.

In addition, the total amount of quaternary compounds exceeds the maximum permissible value of 0.13 wt.-%, and, thus, is not conform with the cosmetic directive.

As can be concluded from the above Example 1 and the Comparative Examples 1 and 2, only the composition according to the present invention which is a combination of at least one quaternary compound according to general formula (I) or formula (II), at least on alcohol or polyol, at least one inorganic or organic acid and at least one essential oil fulfils the EU biocide product directive and the cosmetic directive, i.e. a maximum permissible value for the quaternary compound of less than 0.13 wt.-%.

Hence, only with the combination composition according to the present invention, an antimicrobial effect against a broad spectrum of microbes can be achieved while at the same time the total amount of quaternary compounds can be diminished to the maximum permissible value.

### Comparative Example 3: Quantitative Suspension Test for the Evaluation of Bactericidal Activity According to DIN EN 1276 and DIN EN 1650 Of a One-for-all Hand Sanitizer According to the Prior Art

The tested hand sanitizer had the following composition:

TABLE 5

| Ingredient | Amount |
|---|---|
| (INCI) | (wt.-%) |
| AQUA (WATER) | 99.038691 |
| ROSMARINUS OFFICINALIS (ROSEMARY) OIL | 0.09395 |
| TOCOPHEROL | 0.0007 |
| GLYCINE SOJA OIL UNSAPONIFIABLES | 0.0002 |
| HELIANTHUS ANNUUS (SUNFLOWER) SEED OIL | 0.0001 |
| MENTHA PIPERITA (PEPPERMINT) OIL | 0.04955 |
| MELALEUCA ALTERNIFOLIA (TEA TREE) LEAF OIL | 0.01896 |
| LIMONENE | 0.00655 |
| LINALOOL | 0.0022 |
| DODECYLDIMETHYL[3- | 0.3 |
| (TRIETHOXYSILYL)PROPYL]AMMONIUM | |
| CHLORIDE | |
| N1-OCTADECYL-N1,N1,N2,N2-TETRAMETHYL- | 0.1 |
| N2-(3-(TRIHYDROXYSILYL)PROPYL)ETHANE-1,2- | |
| DIAMINIUM CHLORIDE | |
| ALCOHOL | 0.15624 |
| ASCORBIC ACID | 0.03 |
| UREA | 0.02 |
| CITRIC ACID | 0.18285905 |
| Total: | 100.00 |

The hand sanitizer was tested in concentrations of 80%, 50% and 10% in the same manner as described for the hand sanitizer in Example 1 with the exception that the duration of action was 1 minute.

However, the hand sanitizer did neither fulfil the EU biocide product directive DIN EN 1276 nor the EU biocide product directive DIN EU 1650. The hand sanitizer exhibited an insufficient biocide efficacy, since it is only effective against enveloped viruses, but not against bacterial and least of all against yeast and mould.

In addition, the total amount of quaternary compounds with 0.40 wt.-% is by far higher than the maximum permissible value for quaternary compounds in cosmetic preparations.

### Comparative Example 4: Quantitative Suspension Test for the Evaluation of Bactericidal Activity According to DIN EN 1276 and DIN EN 1650 of a One-for-all Hand Sanitizer According to the Prior Art

The tested hand sanitizer had the following composition:

TABLE 6

| Ingredient | Amount |
|---|---|
| (INCI) | (wt.-%) |
| AQUA (WATER) | 98.899631 |
| ROSMARINUS OFFICINALIS (ROSEMARY) OIL | 0.09395 |
| TOCOPHEROL | 0.0007 |
| GLYCINE SOJA OIL UNSAPONIFIABLES | 0.0002 |
| HELIANTHUS ANNUUS (SUNFLOWER) SEED OIL | 0.0001 |
| MENTHA PIPERITA (PEPPERMINT) OIL | 0.04955 |
| MELALEUCA ALTERNIFOLIA (TEA TREE) LEAF OIL | 0.01896 |
| LIMONENE | 0.00655 |
| LINALOOL | 0.0022 |
| DODECYLDIMETHYL[3- | 0.3 |
| (TRIETHOXYSILYL)PROPYL]AMMONIUM | |
| CHLORIDE | |
| N1-OCTADECYL-N1,N1,N2,N2-TETRAMETHYL- | 0.2 |
| N2-(3-(TRIHYDROXYSILYL)PROPYL)ETHANE- | |
| 1,2-DIAMINIUM CHLORIDE | |
| ALCOHOL | 0.1953 |
| ASCORBIC ACID | 0.03 |
| UREA | 0.02 |
| CITRIC ACID | 0.18285905 |
| Total: | 100.00 |

The hand sanitizer was tested in concentrations of 80%, 50% and 10% in the same manner as described for the hand sanitizer in Example 1 with the exception that the duration of action was 1 minute.

However, the hand sanitizer only fulfilled the EU biocide product directive DIN EN 1276 but not the EU biocide product directive DIN EU 1650. The hand sanitizer exhibited an insufficient biocide efficacy, since it is only effective against bacteria and enveloped viruses, but not against yeast and mould. The efficacy against bacteria is due to the higher concentration of total quaternary compounds (0.50 wt.-%).

In addition, the total amount of quaternary compounds with 0.50 wt.-% is by far higher than the maximum permissible value for quaternary compounds in cosmetic preparations.

### Comparative Example 5: Quantitative Suspension Test for the Evaluation of Bactericidal Activity According to DIN EN 1276 and DIN EN 1650 of a Surface Disinfectant According to the Prior Art

The tested surface disinfectant had the following composition:

TABLE 7

| Ingredient | Amount |
|---|---|
| (INCI) | (wt.-%) |
| AQUA (WATER) | 99.221841 |
| DODECYLDIMETHYL[3- | 0.3 |
| (TRIETHOXYSILYL)PROPYL]AMMONIUM | |
| CHLORIDE | |
| N1-OCTADECYL-N1,N1,N2,N2-TETRAMETHYL- | 0.1 |
| N2-(3-(TRIHYDROXYSILYL)PROPYL)ETHANE- | |
| 1,2-DIAMINIUM CHLORIDE | |
| ALCOHOL | 0.1953 |
| CITRIC ACID | 0.18285905 |
| Total: | 100.00 |

The surface disinfectant was tested in concentrations of 80%, 50% and 10% in the same manner as described for the hand sanitizer in Example 1 with the exception that the duration of action was 1 minute.

However, the surface disinfectant did neither fulfil the EU biocide product directive DIN EN 1276 nor the EU biocide product directive DIN EU 1650. The hand sanitizer exhibited an insufficient biocide efficacy, since it is only effective against enveloped viruses, but not against bacterial and least of all against yeast and mould.

In addition, the total amount of quaternary compounds with 0.40 wt.-% is by far higher than the maximum permissible value for quaternary compounds in cosmetic preparations.

Regarding all three formulations according to the prior art, it can be derived that these formulations do not exhibit a sufficient bactericidal, yeasticidal and fugicidal efficacy.

From the above results it can be concluded that only the composition according to the present invention which is a combination of at least one quaternary compound according to general formula (I) or formula (II), at least on alcohol or polyol, at least one inorganic or organic acid and at least one essential oil fulfils the EU biocide product directives DIN EN 1276 and DIN EN 1650, i.e. the composition has an antimicrobial efficacy, including antibacterial, antiviral, yeasticidal and fugicidal efficacy.

### Example 2: Efficacy Test Against the SARS-CoV-2 Virus

The tested water-based sanitizer had the same formula as described in Example 1, Table 1.

Surface test: Prior to contamination, the sanitizer was applied on smooth leather.

The first-round contamination with a high viral load of SARS-CoV-2 was conducted on the leather surface. 5 min after the contamination, the surface was swabbed for PCR test. The PCR test results was CT value=35. 24 hours later, the second round of contamination with a high viral load of SARS-CoV-2 was conducted on the same area. 5 min after the second round of contamination, the same spot was swabbed for PCR test. The PCR test result was CT value=37.

The suspension test was repeated after the sanitizer/disinfectant solution was diluted to 80% to simulate the more real-world scenario.

Duration of action: 15 s: CT=35; and 30 s: CT=37.

### Example 3: Efficacy Test Against the SARS-CoV-2 Virus

The tested water-based surface disinfectant had the same formula as described in Example 1, Table 1.

Test 1-1: Prior to contamination, the disinfectant was applied on a cylinder-shaped steel surface. The first-round contamination with a high viral load of SARS-CoV-2 was conducted on the round steel surface. 5 min after the contamination, the surface was swabbed for PCR test. The PCR test result was CT value=35. 6 hours later, the second round of contamination with a high viral load of SARS-CoV-2 was conducted on the same area. 5 min after the second round of contamination, the same spot was swabbed for PCR test. The PCR test result was CT value=35. 48 hours later, the third round of contamination with a high viral load of SARS-CoV-2 was conducted on the same area. 5 min after the 3rd round of contamination, the same spot was swabbed for PCR test. The PCR test result was CT value=37.

Test 1-2: Repeated contaminations at 6 h, 24 h, and 48 h, then swabbed after 5 min for PCR test. CT=37.

Test 1-3: A clean cylinder-shaped steel surface was first contaminated with a high viral load of SARS-CoV-2. Subsequently, the disinfectant was applied on the surface, the surface was swabbed for PCR test 15 seconds after applying the disinfectant. The PCR test result was CT value=35. After 6 hours of drying, the surface was contaminated again. 48 hours later, the surface was contaminated again. After 5 min, the same contaminated spot was swabbed for PCR test. The PCR test result was CT value >45; above the detectable range.

The suspension test was repeated after the disinfectant was diluted to 80% to simulate the more real-world scenario.

Duration of action: 15 s: CT=34; and 30 s: CT=37

A low CT value (CT value <25) is generally interpreted to correspond to a high viral load with a high risk of transmission, whereas a high CT value >30 is interpreted as a low risk of transmission. CT >32 to 35 are considered being no risk of transmission, i.e., the viral load is too low to have a pathogenic effect.

As demonstrated by the above examples and comparative examples, the composition according to the present invention is particularly beneficial for killing a broad spectrum of microbes, including gram-positive bacteria, gram-negative bacteria, viruses, non-enveloped and enveloped viruses, mould, mildew, yeast, fungi, and parasites, in one formulation.

### Example 4: Preparation of N1-hexadecyl-N1,N1,N2,N2-tetramethyl-ethane-1-diaminium Chloride (First Reaction Step)

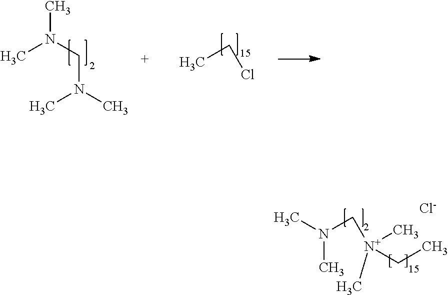

TMEDA (CAS: 110-18-9; 116.2 g/mol) was reacted with 1-chlor-hexadecane (CAS: 4860-03-1; 260.89 g/mol) to give N1-hexadecyl-N1,N1,N2,N2-tetramethyl-ethane-1-diaminium chloride (mono-cation) (377.09 g/mol).

### Example 5: Preparation of N1-octadecyl-N1,N1,N2,N2-tetramethyl-ethane-1-diaminium chloride (First Reaction Step)

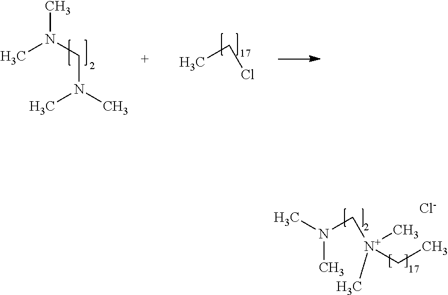

TMEDA (CAS: 110-18-9; 116.2 g/mol) was reacted with 1-chlor-octadecane (CAS: 3386-33-2; 288.9 g/mol) to give N1-octadecyl-N1,N1,N2,N2-tetramethyl-ethane-1-diaminiumchloride (mono-cation) (405.1 g/mol).

### Example 6: Preparation of N1-hexadecyl-N1,N1,N2,N2-tetramethyl-N2-(3-(trihydroxysilyl-)propyl-)ethane-1,2-diaminium Chloride (Second Reaction Step)

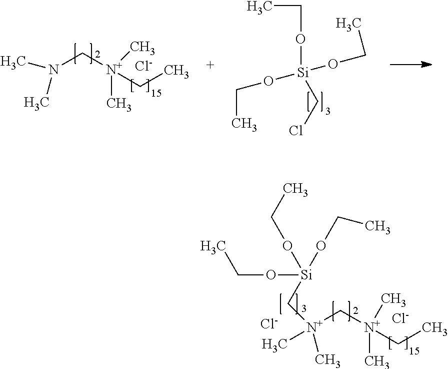

N1-hexadecyl-N1,N1,N2,N2-tetramethyl-ethane-1-diaminium chloride (mono-cation) (377.09 g/mol) from Example 4 was reacted with (3-chloropropyl)triethoxysilane (CAS: 5089-70-3; 240.80 g/mol) to give N1-hexadecyl-N1,N1,N2,N2-tetramethyl-N2-(3-(trihydroxysilyl-)propyl-)ethane-1,2-diaminium chloride (CAS: 2660241-71-2; 617.89 g/mol)

### Example 7: Preparation of N1-octacecyl-N1,N1,N2,N2-tetramethyl-N2-(3-(trihydroxysilyl-)propyl-)ethane-1,2-diaminium Chloride (Second Reaction Step)

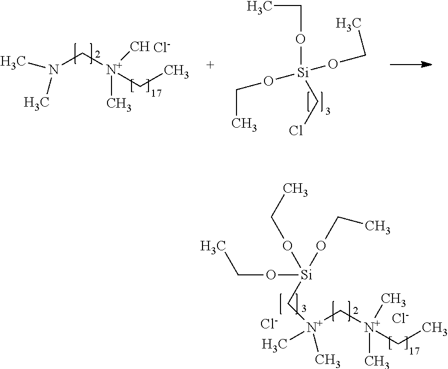

N1-octadecyl-N1,N1,N2,N2-tetramethyl-ethane-1-diaminiumchloride (mono-cation) (405.1 g/mol) from Example 5 was reacted with (3-Chloropropyl)triethoxysilane (CAS: 5089-70-3; 240.80 g/mol) to give N1-octacecyl-N1,N1,N2,N2-tetramethyl-N2-(3-(trihydroxysilyl-)propyl-)ethane-1,2-diaminium chloride (CAS: 2660241-69-8; 645.90 g/mol).

1. A composition comprising:
(a) at least one quaternary compound represented by the general formula (I) 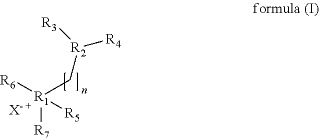
wherein
R₁ and R₂ are independently from each other selected from the group consisting of nitrogen and phosphor;
R₃ and R₄ are independently from each other selected from the group consisting of H, C1 to C36 n-alkyl, C1 to C36 i-alkyl, C1 to C36 sec-alkyl, C1 to C36 t-alkyl, aryl or C1 to C36 t-alkyl-silyl;
R₅ and R₆ are independently from each other selected from the group consisting of H, C1 to C36 n-alkyl, C1 to C36 i-alkyl, C1 to C36 sec-alkyl, C1 to C36 t-alkyl, aryl and C1 to C36 t-alkyl-silyl;
R₇ is a C1 to C36 n-alkyl;
X is selected from the group consisting of Cl, Br, F, I, HSO₄, 0.5HPO₄, H₂PO₄, 0.5CO₃, 0.5SO₄, ⅓PO₄, CH₃SO₃, CF₃SO₃,NTf₂, BF₄,acetate, tosylate, benzoate, salicylate, succinate, saccharinate, —OH and gluconate; and
n is an integer from 1 to 36;
or at least one quaternary compound represented by the general formula (II) 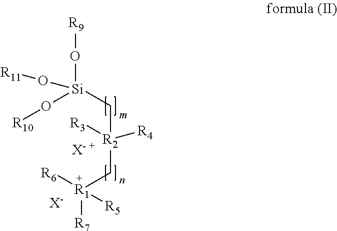
wherein
R₁ and R₂ are independently from each other selected from the group consisting of nitrogen and phosphor;
R₃ and R₄ are independently from each other selected from the group consisting of H, C1 to C36 n-alkyl, C1 to C36 i-alkyl, C1 to C36 sec-alkyl, C1 to C36 t-alkyl, aryl or C1 to C36 t-alkyl-silyl;
R₅ and R₆ are independently from each other selected from the group consisting of H, C1 to C36 n-alkyl, C1 to C36 i-alkyl, C1 to C36 sec-alkyl, C1 to C36 t-alkyl, aryl and C1 to C36 t-alkyl-silyl;
R₇ is a C1 to C36 n-alkyl;
X is selected from the group consisting of Cl, Br, F, I, HSO₄, 0.5HPO₄, H₂PO₄, 0.5CO₃, 0.5SO₄, ⅓PO₄, CH₃SO₃, CF₃SO₃,NTf₂, BF₄,acetate, tosylate, benzoate, salicylate, succinate, saccharinate, —OH and gluconate;
R₉, R₁₀ and R₁₁ are independently from each other selected from the group consisting of H, C1 to C36 n-alkyl, C1 to C36 i-alkyl, C1 to C36 sec-alkyl, C1 to C36 t-alkyl and aryl;
n is an integer from 1 to 36; and
m is an integer from 1 to 36;
(b) at least one alcohol and/or polyol;
(c) at least one inorganic and/or organic acid or a salt or a derivative thereof, and
(d) at least one essential oil.

2. The composition according to claim 1, wherein in general formula (I)
R₁ and R₂ are independently from each other selected from the group consisting of nitrogen and phosphor; and/or
R₃ and R₄ are independently from each other selected from the group consisting of H—, methyl, ethyl, n-propyl, i-propyl, n-butyl and sec-butyl; and/or
R₅ and R₆ are independently from each other selected from the group consisting of H—, methyl, ethyl, n-propyl, i-propyl n-butyl and sec-butyl; and/or
R₇ is a C1 to C36 n-alkyl; and/or
X is selected from the group consisting of Cl, Br, F, I, HSO₄, 0.5HPO₄, H₂PO₄, 0.5CO₃, 0.5SO₄, ⅓PO₄, CH₃SO₃, CF₃SO₃, NTf₂, BF₄, acetate, tosylate, benzoate, salicylate, succinate, saccharinate, —OH and gluconate; and/or
n is an integer from 1 to 36
or wherein in general formula (II)
R₁ and R₂ are independently from each other selected from the group consisting of nitrogen and phosphor, and/or
R₃ and R₄ are independently from each other selected from the group consisting of H—, methyl, ethyl, n-propyl, i-propyl, n-butyl and sec-butyl; and/or
R₅ and R₆ are independently from each other selected from the group consisting of H—, methyl, ethyl, n-propyl, i-propyl n-butyl and sec-butyl;
R₇ is a C1 to C36 n-alkyl; and/or
X is selected from the group consisting of Cl, Br, F, I, HSO₄, 0.5HPO₄, H₂PO₄, 0.5CO₃, 0.5SO₄, ⅓PO₄, CH₃SO₃, CF₃SO₃, NTf₂, BF₄, acetate, tosylate, benzoate, salicylate, succinate, saccharinate, —OH and gluconate;
R₉, R₁₀ and R₁₁ are independently from each other selected from the group consisting of H—, methyl, ethyl, n-propyl and n-butyl; and/or
n is an integer from 1 to 36; and/or
m is an integer from 1 to 36.

3. The composition according to claim 1, wherein the at least one quaternary compound is selected from the group consisting of
N1-hexadecyl-N1,N1,N2,N2-tetramethyl-ethane-1-diaminium chloride;
N1-octacecyl-N1,N1,N2,N2-tetramethyl-ethane-1-diaminium chloride;
N1-hexadecyl-N1,N1,N2,N2-tetramethyl-N2-(3-(trihydroxysilyl-)propyl-)ethane-1,2-diaminium chloride;
N1-octacecyl-N1,N1,N2,N2-tetramethyl-N2-(3-(trihydroxysilyl-)propyl-)ethane-1,2-diaminium chloride;
and any mixture thereof.

4. The composition according to claim 1, wherein the composition comprises at least two quaternary compounds according to the general formula (I) and/or general formula (II).

5. The composition according to claim 1, wherein the composition comprises at least four quaternary compounds according to the general formula (I) and/or general formula (II).

6. The composition according to claim 1, wherein the composition comprises a mixture of
N1-hexadecyl-N1,N1,N2,N2-tetramethyl-ethane-1-diaminium chloride;
N1-octadecyl-N1,N1,N2,N2-tetramethyl-ethane-1-diaminium chloride;
N1-hexadecyl-N1,N1,N2,N2-tetramethyl-N2-(3-(trihydroxysilyl-)propyl-)ethane-1,2-diaminium chloride;
N1-octacecyl-N1,N1,N2,N2-tetramethyl-N2-(3-(trihydroxysilyl-)propyl-)ethane-1,2-diaminium chloride.

7. The composition according to claim 1, wherein the at least one alcohol and/or polyol component (b) is selected from the group consisting of ethanol, n-propanol, 2-propanol, n-butanol, 2-butanol, 3-butanol, tert.-butanol, 2-methyl-1-butanol, 3-methyl-1-butanol, 3-methyl-2-butanol, 3-methoxy-3-methyl-1-butanol, 2-methyl-2-butanol, 3,3-dimethylbutane-2-ol, 2,2-dimethyl-1-propanol, hexane-1-ol, hexane-2-ol, hexane-3-ol, 2-methylpentane-1-ol, 2-methylpentane-2-ol, 2-methylpentane-3-ol, 4-methylpentane-1-ol, 4-methylpentane-2-ol, 3-methylpentane-1-ol, 3-methylpentane-2-ol, 3-methylpentane-3-ol, 2,2-dimethylbutane-1-ol, 3,3-dimethylbutane-1-ol, 3,3-dimethylbutane-2-ol, 2,3-dimethylbutane-2-ol, 2,3-dimethylbutane-1-ol, 2-ethylbutane-1-ol, glycerol, propylene glycol, butylene glycol, pentylene glycol, hexylene glycol, pentaerythritol, trimethylolpropane, 1,2-ethandiol, 1,4,6-octanetriol, propanediol, butanediol, pentanediol, hexanediol, octanediol, chloropentanediol, glycerol monoalkyl ether, glycerol monoethyl ether, diethylene glycol, dipropylene glycol, 2-ethylhexanediol-1,4, cyclohexanediol-1,4, 1,2,6-hexanetriol, 1,3,5-hexanetriol, 1,2,3-hexanetriol, 1,3-bis-(2-hydroxyethoxy)propane, heptane-2,4,6-triol, heptane-1,2,3-triol, heptane-1,4,7-triol, heptane-1,2,7-triol, neopentyl glycol, 2-methyl propanediol, trimethylol propane monoallylether, 2,2,4-trimethyl-1,3-pentanediol, 1,4-butanediol, cyclohexanedimethanol, and 2,2-dimethyl-3-hydroxypropyl-2,2-dimethyl-3-hydroxypropionate, phenoxyethanol, hexylresorcinol, phenethylalcohol, benzylalcohol and any mixture thereof.

8. The composition according to claim 1, wherein the at least one inorganic and/or organic acid component (c) is selected from the group consisting of salicylic acid, sorbic acid, lactic acid, hydrochloric acid, boric acid, sulfuric acid, hyposulfuric acid, phosphoric acid, perchloric acid, peroxymonosulfuric acid, peroxydisulfuric acid, hypodiphosphonic acid, hypodiphosphoric acid, diphosphonic acid, diphosphoric acid, benzoic acid, anisic acid, formic acid, butanoic acid, propionic acid, citric acid, acetic acid, acetylsalicylic acid, ascorbic acid, malic acid, tartaric acid, gallic acid, glyoxylic acid, fumaric acid, glycolic acid, ferulic acid, mandelic acid, acrylic acid, malonic acid, hypophosphorous acid/phosphinic acid, sulfurous acid, peroxodiphosphoric acid, artemisinic acid, peroxophosphoric acid, permanganic acid, undecylenic acid, levulinic acid, 1-naphthylacetic acid, hydroxamic acid, caprylic acid, caproic acid, adipic acid, gamma-hydroxybutyric acid, capric acid, ellagic acid, usnic acid, ricinoleic acid, quinic acid, chorismic acid, isophthalic acid, oxaloacetic acid, squaric acid, oleic acid, bicinchoninic acid, barbituric acid, citronellic acid, crotonic acid, isocrotonic acid, vinyl acetic acid, succinic acid, shikimic acid, maleic acid, valeric acid, clavulanic acid, alendronic acid, arachic acid, arachidonic acid, behenic acid, hydrocyanic acid, pyruvic acid, carbamic acid, linoleic acid, alpha-linolenic acid, cerotic acid, fluor acetic acid, chloro acetic acid, bromo acetic acid, Iodo acetic acid, peracetic acid, glutamic acid, aspartic acid, methanesulfonic acid, erucic acid, fusaric acid, fusidic acid, gamma-aminobutyric acid, gondosoic acid, glucaric acid, gluconic acid, lauric acid, lignoceric acid, margaric acid, melissic acid, montanic acid, myristic acid, nermonic acid, oxaacetic acid, palmitoleic acid, linseedic acid, thiosulfuric acid, trichloroacetic acid, trifluoromethanesulfuric acid, trifluoroacetic acid, abietic acid, cinnamic acid, vulpic acid, tetrahydrofolic acid, phthalic acid, uric acid, fusidic acid, phenylacetic acid, N-acetlyneuramic acid, indole-3-acetic acid, ibotenic acid, hippuric acid, folic acid, terephthalic acid, sulphanilic acid, styphnic acid, picric acid, heptanoic acid, pimelic acid, suberic acid, azelaic acid, sebacic acid, pelargonic acid, isocitric acid, aconitic acid, allylacetic acid, glutaric acid, alpha-ketoglutaric acid, 2,5-dihydroxybenzoic acid, itaconic acid, perillaic acid, 3,4-dihydroxybenzoic acid, capryloyl glycine, PEG-6 undecylenate, propane caprylate, undecylenoyl glycine, undecylenoyl sarcosine, zink glycinate salicylate, amino-levulinic acid-hydrochlorid, biphenyl hydroxamic acid, C12-C15 pareth-3-benzoate, capryloyl methyl serinate, capryloyl serinate, capryloyl salicylic acid, caprylyl glycol linseedate, ricinoleic acid benzoate, cetyl ricinoleic acid benzoate, sorbityl dimonium anisate (CAS-No.: 2097714-04-8, zink cysteiante, any mixture thereof, and salts and derivatives of the afore-mentioned acids.

9. The composition according to claim 1, wherein the at least one essential oil component (d) is selected from the group consisting of Mentha piperita (peppermint) oil (CAS-No: 8006-90-4), Mentha spicata oil, Salvia sclarea oil, Fragaria ananassa seed oil, Rosmarinus officinalis oil (CAS-No: 8000-25-7), Origanum oil (CAS-No.: 8007-11-2), Achillea millefolium oil, Nageia nagi oil, Peumus boldus oil, Salvia officinalis oil (CAS-No: 8022-56-8), Sorbus aucuparia seed oil, Wrightia tinctoria oil, Abies alba oil, Abies pectinata oil, Adenosma nelsonioides oil, Aeollanthus, Bakuchiol suaveolens oil, Aframomum angustifolium oil, Aleurites moluccanus bakoly oil, alligator oil, Allium cepa bulb oil, Artemisia absinthium oil, Bakuchiol, Calophyllum inophyllum oil, Cannabis sativa oil, Camellia sinensis oil, Chamaecyparis formosensis oil, Cinnamomum cassia bark oil, Cinnamosma fragrans oil, Clausena lansium oil, Commiphora gileadensis oil, Cyperus scariosus rhizome oil, Dendropanax melaleuca Alternifolia oil (CAS-No: 68647-73-4), Morbiferus stem sap oil, Dracocephalum foetidum oil, Dracocephalum moldavica oil, Eugenia uniflora oil, Ferula galbaniflua resin oil, Hippophae rhamnoides oil, hydrogenated Nepeta cataria oil, Leptospermum laevigatum oil, Lindera umbellata oil, Lippia origanoides oil, Mentha suaveolens oil, Pistacia lenticus gum oil (CAS-No: 61789-92-2), Pistacia lenticus leaf oil (CAS-No: 90082-82-9), Psiadia altissima leaf oil, Salvia triloba oil, Sapindus mukorossi oil, Schinus terebinthifolia oil, Schizochytrium limacinum oil, Tagetes lemmonii oil, Thujopsis dolabrata oil, Thymus vulgaris oil (CAS-No: 84929-51-1), Torreya nucifera leaf oil (CAS-No: 84929-51-1), Ocimum basilicum oil (CAS-No.: 84775-71-3), Pistacia lenticus gum, and any mixture thereof.

10. The composition according to claim 1, wherein the composition further comprises
(e) at least one antimicrobial component which is different from the at least one quaternary component (a); and/or
(f) at least one quaternary organic nitrogen or phosphorous containing component; and/or
(g) at least one tenside component.

11. The composition according to claim 1, wherein the composition further comprises
(h) a solvent; and/or
(i) a film forming material.

12. The composition according to claim 1, wherein the composition comprises:
(a) 0.001 to 2 wt.-% of the at least one quaternary compound;
(b) 0.0001 to 15 wt.-% of the at least one alcohol and/or polyol component;
(c) 0.0001 to 4 wt.-% of the at least one inorganic and/or organic acid component; and
(d) 0.0001 to 5 wt.-% of the at least one essential oil component,
based on the total weight of the composition.

13. A disinfectant or antimicrobial preparation comprising the composition according to claim 1.

14. (canceled)

15. The disinfectant or antimicrobial preparation of claim 13, wherein the preparation is active against microorganisms selected from the group consisting of gram-positive bacteria, gram-negative bacteria, viruses, non-enveloped and enveloped viruses, mould, mildew, yeast, fungi and parasites.

16. A method for disinfecting a surface, skin or an object comprising the steps of:
applying an effective amount of the composition of claim 1 onto the surface, skin or object; and
contacting the surface, skin or the object with the composition for a contact time of at least 15 seconds.

17. Method for preparing a quaternary compound according to general formula (I) as defined in claim 1, comprising the steps of
mixing Y1,Y1,Y2,Y2-tetra-(alkyl-/aryl-)alkylendipnictogenin with a n-halogen alkane,
heating the reaction mixture to a temperature of at least 60° C.;
letting the mixture react for at least 4 days at a temperature of at least 60° C.; and
separating the Y1-(alkyl-/aryl-)Y1,Y1,Y2,Y2-tetra-(alkyl-/aryl-)alkane-1-dipnictogeninium halogenide;
according to the following reaction schemata: 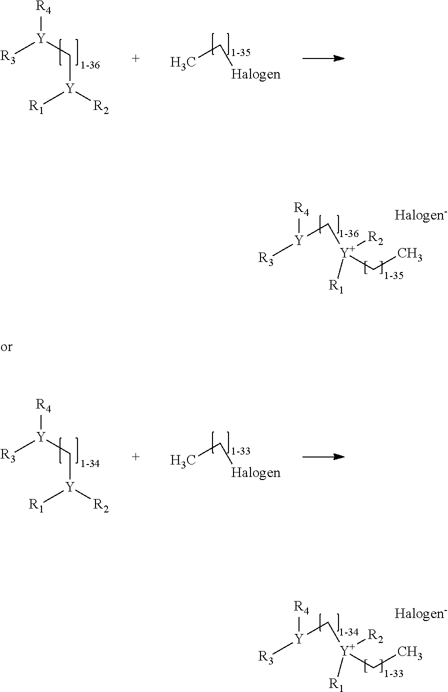
wherein
Y represents phosphor or nitrogen;
R₁ and R₂ are independently from each other selected from the group consisting of H, C1 to C36 n-alkyl, C1 to C36 i-alkyl, C1 to C36 sec-alkyl, C1 to C36 t-alkyl, aryl or C1 to C36 t-alkyl-silyl;
R₃ and R₄ are independently from each other selected from the group consisting of H, C1 to C36 n-alkyl, C1 to C36 i-alkyl, C1 to C36 sec-alkyl, C1 to C36 t-alkyl, aryl and C1 to C36 t-alkyl-silyl; and
halogen is selected from the group consisting of F, Cl, Br and I.

18. Method for preparing a quaternary compound according to the general formula (II) as defined in claim 1, comprising the steps of
mixing Y1-(alkyl-/aryl-)Y1,Y1,Y2,Y2-tetra-(alkyl-/aryl-)alkane-1-dipnictogeninium halogenide with a n-halogenoalkyl trialkoxysilan;
heating the reaction mixture to a temperature of at least 60° C.;
letting the mixture react for at least 4 days at a temperature of at least 60° C.; and
separating the Y1-(alkyl-/aryl-)Y1,Y1,Y2,Y2-tetra-(alkyl-/aryl-)-Y2-(3-[tri(alkyloxy-/hydroxy-)silyl-]alkyl)alkane-1,2-dipnictogeninium halogenide;
according to the following reaction schemata: 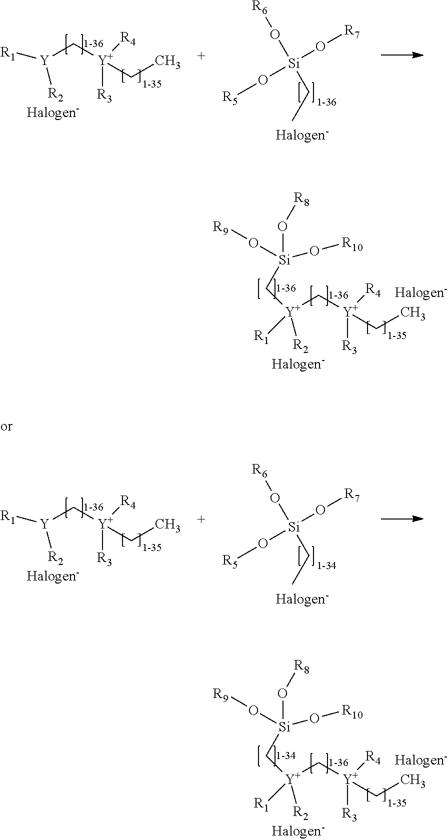
wherein
Y represents phosphor or nitrogen;
R₁ and R₂ are independently from each other selected from the group consisting of H, C1 to C36 n-alkyl, C1 to C36 i-alkyl, C1 to C36 sec-alkyl, C1 to C36 t-alkyl, aryl or C1 to C36 t-alkyl-silyl;
R₃ and R₄ are independently from each other selected from the group consisting of H, C1 to C36 n-alkyl, C1 to C36 i-alkyl, C1 to C36 sec-alkyl, C1 to C36 t-alkyl, aryl and C1 to C36 t-alkyl-silyl;
R5, R6 and R7 are independently from each other selected from the group consisting of H, C1 to C36 n-alkyl, C1 to C36 i-alkyl, C1 to C36 sec-alkyl, C1 to C36 t-alkyl and aryl; and
halogen is selected from the group consisting of F, Cl, Br and I.

19. The composition according to claim 1, wherein in general formula (I)
R₁ and R₂ are independently from each other selected from the group consisting of nitrogen and phosphor; and/or
R₃ and R₄ are independently from each other selected from the group consisting of methyl, ethyl, n-propyl, i-propyl and n-butyl; and/or
R₅ and R₆ are independently from each other selected from the group consisting of methyl, ethyl and n-propyl; and/or
R₇ is a C6 to C24 n-alkyl; and/or
X is selected from the group consisting of Cl, Br, F and I; and/or
n is an integer from 10 to 20;
or wherein in general formula (II)
R₁ and R₂ are nitrogen; and/or
R₃ and R₄ are independently from each other selected from the group consisting of methyl, ethyl, n-propyl and n-butyl; and/or
R₅ and R₆ are independently from each other selected from the group consisting of methyl, ethyl, n-propyl and n-butyl; and/or
R₇ is a C6 to C24 n-alkyl; and/or
X is selected from the group consisting of Cl, Br, F and I; and/or
R₉, R₁₀ and R₁₁ are independently from each other selected from the group consisting of H—, methyl, ethyl and n-propyl; and/or
n is an integer from 10 to 20; and/or
m is an integer from 10 to 20.

20. The composition according to claim 1, wherein in general formula (I)
R₁ and R₂ are nitrogen; and/or
R₃ and R₄ are independently from each other selected from the group consisting of methyl and ethyl; and/or
R₅ and R₆ are independently from each other selected from the group consisting of methyl and ethyl; and/or
R₇ is a C10 to C20 n-alkyl; and/or
X is selected from the group consisting of Cl and Br; and/or
n is an integer from 12 to 18;
or wherein in general formula (II)
R₁ and R₂ are nitrogen; and/or
R₃ and R₄ are independently from each other selected from the group consisting of methyl and ethyl; and/or
R₅ and R₆ are independently from each other selected from the group consisting of methyl and ethyl; and/or
R₇ is a C10 to C20 n-alkyl; and/or
X is selected from the group consisting of Cl and Br; and/or
R₉, R₁₀ and R₁₁ are independently from each other selected from the group consisting of H— and ethyl; and/or
n is an integer from 12 to 18 and/or
m is an integer from 12 to 18.

21. The method of claim 16, wherein the contact time is at least 30 minutes.
